(19) United States
(12) Patent Application Publication
(10) Pub. No.: US 2012/0076797 A1
(43) Pub. Date: Mar. 29, 2012

(54) Modulation of kit signaling and hematopoietic cell development by IL-4 receptor modulation

(76) Inventors: Atul J. Butte, Stanford, CA (US); Kenneth Weinberg, Stanford, CA (US); Shivkumar Venkatasubrahmanyam, San Jose, CA (US); Maheswaran Mani, Sunnyvale, CA (US)

(21) Appl. No.: 13/135,654

(22) Filed: Jul. 12, 2011

Related U.S. Application Data
(60) Provisional application No. 61/364,363, filed on Jul. 14, 2010.
(60) Provisional application No. 61/399,783, filed on Jul. 15, 2010.

(51) Int. Cl. A61K 39/395; C12Q 1/02; C12Q 1/06; A61P 35/00; A61P 35/02; A61P 7/06; G01N 33/566; C12Q 1/68

(52) U.S. Cl. 424/158.1; 435/7.21; 435/29; 435/6.12; 435/39

(57) ABSTRACT

Methods and compositions are provided for modulating Kit/stem cell factor receptor (SCFR)/CD117 and interleukin 4 receptor (IL-4R) signaling in a cell in vitro and in vivo, and for identifying candidate agents with activity in modulating Kit and IL-4R signaling. These methods find particular use in treating disorders of the hematopoietic system and in modulating hematopoietic stem cell expansion.

### GOVERNMENT RIGHTS

This invention was made with government support under R01CA138256 and P01 CA049605-20 awarded by the National Cancer Institute, R01A1050765 awarded by the National Institute of Allergy and Infectious Disease, and LM009719, CA138256, CA049605, and A1050765 awarded by the National Institutes of Health. The Government has certain rights in the invention.

### FIELD OF THE INVENTION

This invention pertains to methods and composition for modulating the signaling of the cell surface receptors Kit and IL-4R, and the use of such methods and compositions in the treatment of disease.

### BACKGROUND OF THE INVENTION

Expression of the receptor tyrosine kinase Kit/stem cell factor receptor (SCFR)/CD117 is a hallmark of embryonic stem cells (ESC) and many tissue-specific adult stem cells, pointing to a central role for Kit signaling in stem cell biology. Stem cell populations that express Kit and that are regulated by Kit signaling include embryonic stem (ES) cells (Palmqvist, L. et al. Stem Cells 23, 663-680 (2005)), hematopoietic stem cells (HSC) (Broudy, V. C. Blood 90, 1345-1364 (1997); Lyman, S. D. & Jacobsen, S. E. Blood 91, 1101-1134 (1998)) neural stem cells (NSC) (Erlandsson, A. et al. Exp Cell Res 301, 201-210 (2004); Sun, L. et al. J Clin Invest 113, 1364-1374 (2004)) and cardiac stem cells (CSC) (Beltrami, A. P. et al. Cell 114, 763-776 (2003)). Additionally Kit is expressed by and functions in primitive lymphoid (Palacios, R. & Nishikawa, S. Development 115, 1133-1147 (1992)) and erythroid (Olweus, J. et al. Blood 88, 1594-1607 (1996)) progenitors as well as germ cells (Farini, D. et al. Dev Biol 306, 572-583 (2007); Mauduit, C. et al. Hum Reprod Update 5, 535-545 (1999)) and melanocyte precursors (Ito, M. et al. J Invest Dermatol 112, 796-801 (1999)). Disruption of Kit signaling by mutations in Kit or its ligand or by specific inhibitors results in a spectrum of defects in these stem cell populations and tissues arising from them including defects in ESC survival and differentiation capacity (Bashamboo, A. et al. J Cell Sci 119, 3039-3046 (2006); Lu, M. et al. Exp Hematol 35, 1293-1302 (2007)) macrocytic anemia and pancytopenia (Broudy, V. C. Blood 90, 1345-1364 (1997)), learning and memory defects (Motro, B. et al. Proc Natl Acad Sci USA 93, 1808-1813 (1996)), defects in CSC differentiation (Li, M. et al. Circ Res 102, 677-685 (2008)), sterility (Mauduit, C. et al. Hum Reprod Update 5, 535-545 (1999)) and pigmentation defects (Spritz, R. A. et al. Am J Hum Genet 51, 1058-1065 (1992)). Kit signaling has been extensively studied in hematopoietic cells and has been shown to regulate the survival, proliferation and self-renewal of HSC (Lennartsson, J. et al. Stem Cells 23, 16-43 (2005)), which give rise to all lineages of blood cells.

A general hypothesis to explain the pleiotropic expression and function of Kit in stem and progenitor populations is that Kit activates different cell-surface receptors in each cell type, providing a mechanistic basis for the distinct but overlapping cell type-specific responses to Kit signaling that have been observed (Blume-Jensen, P. et al. Nat Genet 24, 157-162 (2000); Agosti, V. et al. J Exp Med 199, 867-878 (2004); Kimura, Y. et al. Proc Natl Acad Sci USA 101, 6015-6020 (2004)).

Elucidation of novel molecular interactions has become an engine of biological insight in the post-genomics era. However, approaches to discover signal transduction interactions are particularly limited in their scope by the enormous costs and time required for experimental determination and validation. Thus there is a growing need for methods to computationally predict genes and proteins interacting in receptor signaling, for example Kit signaling, which could lead to increased understanding of the factors driving stem cell pluripotency, methods of inducing pluripotency in cells, and methods of treating disease.

The present invention addresses these issues.

### SUMMARY OF THE INVENTION

Methods and compositions are provided for modulating Kit/stem cell factor receptor (SCFR)/CD117 and interleukin 4 receptor (IL-4R) signaling in a cell in vitro and in vivo, and for identifying candidate agents with activity in modulating Kit and IL-4R signaling.

In some aspects of the invention, methods for modulating Kit signaling in a cell are provided. In these methods, a Kit⁺IL-4R⁺ cell is contacted with an effective amount of an IL-4R modulating agent under conditions that promote cell survival and Kit signaling is measured as a function of IL-4R signaling, where an effective amount of an IL-4R modulating agent to reduce IL-4R signaling (relative to IL-4R signaling in a cell that has not been contacted with the modulatory agent) reduces Kit signaling, and an effective amount of an IL-4R modulating agent to promote IL-4R signaling (relative to IL-4R signaling in a cell that has not been contacted with the modulatory agent) promotes Kit signaling.

In some embodiments, the IL-4R modulating agent reduces IL-4R signaling, i.e. is an IL-4R inhibitor, in which case Kit signaling is reduced. In some embodiments, the IL-4R inhibitor is a peptide agent. In certain embodiments, the peptide agent is an IL-4R antibody or soluble IL-4Rα polypeptide. In some embodiments, the IL-4R inhibitor is a nucleic acid agent. In certain embodiments, the nucleic acid agent is an IL-4Rα subunit siRNA or a cDNA encoding recombinant soluble IL-4Rα. In some embodiments, the IL-4R inhibitor is a small molecule. In some embodiments, the contacting step is executed in vitro. In other embodiments, the contacting step is executed in vivo, i.e., in an individual. In some such embodiments, the individual has anemia, neutropenia, monocytopenia, eosinopenia, thrombocytopenia, mastocytosis, lymphoma (e.g., a B-cell lymphoma), or leukemia (e.g., acute lymphoblastic leukemia, (ALL)). In some embodiments, the number of erythrocytes, neutrophils, monocytes, eosinophils, and platelets in the individual is increased and the number of lymphocytes in the individual is decreased relative to the number of erythrocytes, neutrophils, monocytes, eosinophils, platelets, and lymphocytes in the individual prior to the contacting step. In some embodiments, the method further comprises the step of contacting the Kit+IL-4R+ cell with an agent that reduces Kit signaling, i.e., a Kit inhibitor. In some such embodiments, this method provides for enhanced responsiveness to the Kit inhibitor relative to contacting the Kit+IL-4R+ cell with Kit inhibitor in the absence of IL-4R inhibitor.

In some embodiments, the IL-4R modulating agent promotes IL-4R signaling, i.e. is an IL-4R activator, in which case Kit signaling is promoted. In some embodiments, the IL-4R activator is a peptide agent. In certain embodiments, the peptide agent is an IL-4 peptide. In some embodiments, the IL-4R activator is a nucleic acid. In certain embodiments, the nucleic acid agent is a nucleic acid encoding an IL-4 peptide. In some embodiments, the IL-4R activator is a small molecule. In some embodiments, the contacting step is executed in vitro. In some embodiments, the contacting step is executed in vivo, i.e., in an individual. In some such embodiments, the individual has polycythemia, an infection, atopy, and/or lymphocytopenia. In some embodiments, the number of erythrocytes, neutrophils, monocytes, eosinophils, and platelets in the individual is decreased and the number of lymphocytes in the individual is increased relative to the numbers of erythrocytes, neutrophils, monocytes, eosinophils, platelets, and lymphocytes in the individual prior to the contacting step. In some embodiments, the method further comprises the step of contacting the Kit+IL-4R+ cell with an agent that promotes Kit signaling, i.e., a Kit activator. In some such embodiments, this method provides for enhanced responsiveness to the Kit activator relative to contacting the Kit+IL-4R+ cell with Kit activator in the absence of IL-4R activator.

In some aspects of the invention, methods are provided for modulating IL-4R signaling in a cell. In these methods, a Kit+IL-4R+ cell is contacted with an effective amount of a Kit modulating agent and IL-4R signaling is measured, where an effective amount of Kit modulating agent to reduce Kit signaling (relative to Kit signaling in a Kit+IL-4R+ cell that has not been contacted with the modulatory agent) reduces IL-4R signaling, and an effective amount of Kit modulating agent to promote Kit signaling (relative to Kit signaling in a Kit+IL-4R+ cell that has not been contacted with the modulatory agent) promotes IL-4R signaling.

In some embodiments, the Kit modulating agent reduces Kit signaling, i.e. is a Kit inhibitor, in which case, IL-4R signaling is reduced. In some such embodiments, the Kit inhibitor is a peptide agent. In certain embodiments, the peptide agent is a Kit antibody or Kit extracellular domain polypeptide. In some embodiments, the Kit inhibitor is a nucleic acid agent. In certain embodiments, the nucleic acid agent is a Kit siRNA. In some embodiments, the Kit inhibitor is a small molecule. In certain embodiments, the small molecule is a tyrosine kinase inhibitor. In certain embodiments, the tyrosine kinase inhibitor is Imatinib mesylate/STI571/Gleevac™. In some embodiments, the contacting step is executed in vitro. In other embodiments, the contacting step is executed in vivo, i.e., in an individual. In some such embodiments, the number of erythrocytes, neutrophils, monocytes, eosinophils, and platelets in the individual is increased and the number of lymphocytes in the individual is decreased relative to the numbers of erythrocytes, neutrophils, monocytes, eosinophils, platelets, and lymphocytes in the individual prior to the contacting step.

In some embodiments, the Kit modulating agent promotes Kit signaling, i.e. is a Kit activator, in which case IL-4R signaling is promoted. In some embodiments, the Kit activator is a peptide agent. In certain embodiments, the peptide agent is Kit ligand. In some embodiments, the Kit activator is a nucleic acid. In certain embodiments, the nucleic acid agent is a nucleic acid encoding a Kit ligand. In some embodiments, the Kit activator is a small molecule. In some embodiments, the contacting step is executed in vitro. In other embodiments, the contacting step is executed in vivo, i.e., in an individual. In some such embodiments, the number of erythrocytes, neutrophils, monocytes, eosinophils, and platelets in the individual is decreased and the number of lymphocytes in the individual is increased relative to the numbers of erythrocytes, neutrophils, monocytes, eosinophils, platelets, and lymphocytes in the individual prior to the contacting step.

In some aspects of the invention, methods are provided for enhancing the responsiveness of Kit+IL-4R+ cells to a Kit inhibitor, e.g. to reduce the survival, proliferation, and/or migration of cancer cells. In such methods, Kit+IL-4R+ cells are contacted with an effective amount of a Kit inhibitor and an effective amount of an IL-4R inhibitor under conditions that promote cell survival. In some such embodiments, the method further comprises measuring survival, proliferation, and/or migration of the Kit⁺IL-4R⁺ cells, where survival, proliferation, and/or migration of the Kit⁺IL-4R⁺ cells is reduced relative to survival, proliferation, and/or migration of Kit⁺IL-4R⁺ cells contacted with a Kit inhibitor in the absence of an IL-4R inhibitor. In some embodiments, the method is performed in vivo, that is, in an individual. In some embodiments, the individual has cancer. In some embodiments, the cancer is lymphoma or leukemia.

In some aspects of the invention, methods are provided for enhancing the responsiveness of Kit+IL-4R+ cells to a Kit activator, e.g. to augment the proliferation of cells. In such methods, a population comprising Kit+IL-4R+ cells is contacted with an effective amount of a Kit activator and an effective amount of an IL-4R activator under conditions that promote cell survival. In some embodiments, the method further comprises measuring the number of cells in the population, where the number of cells in the culture is elevated relative to the number of cells in a population contacted with a Kit activator in the absence of an IL-4R activator under the same conditions. In some embodiments, the method is performed in vitro, that is, in cell culture. In some such embodiments, the Kit+IL-4R cells are stem cells.

In some aspects of the invention, methods are provided for screening candidate agents for activity in reducing Kit signaling. In such methods, a population of Kit+IL-4R+ cells is contacted with a candidate agent, and Kit signaling is measured as a function of IL-4R activity, where a reduction in IL-4R activity in the contacted population relative to a Kit+IL-4R+ population that has not be contacted with the candidate agent indicates that the candidate agent is effective in reducing Kit signaling.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Bioinformatics methodology to predict novel Kit-interacting receptors. Predictions of novel Kit-interacting receptors were generated by combining prior knowledge of known receptor interactions (light blue) with publicly available gene expression data (light pink), using co-expression and comparative biology (light purple). The initial list of candidate receptors comprised Type I cytokine receptors, the receptor family to which the two known Kit-interacting receptors, EpoR and IL-7R, belong. A database of gene expression profiles was aggregated from NCBI GEO by manual curation. This database represents a broad range in the expression of Kit across the tissue of interest (HSC) as well as control tissues (lung, kidney and adipocyte). Genes encoding subunits of candidate receptors were clustered hierarchically based on their expression profiles across human and murine samples in our database. Three successive specificity filters were used to identify receptors for which all subunits exhibit coexpression with Kit across multiple tissues and across mammalian species.

FIG. 2. Known Kit-interacting receptor subunits exhibit a Kit-like expression profile across tissues and species. Hierarchical clustering of genes encoding subunits of Type I cytokine receptors and Kit, by gene expression measurements from hematopoietic progenitors (HP) and control tissues (lung, kidney, adipocytes) of mouse (a) or human (b) origin. Rank-normalized mRNA levels for each gene (row) in each sample (column) are shown according to the indicated color scale, with darker color indicating higher relative expression within a sample. Samples are grouped by tissue, as indicated by the banner above each heatmap. Based on manual curation, samples representing human hematopoietic progenitors are further grouped according to high (dark pink) or low (light pink) stringency in HSC selection. This delineation correlates strongly with Kit expression. Genes encoding subunits of EpoR (EPOR) and IL-7R (IL7RA and IL2RG) have expression profiles similar to that of KIT. The smallest clades that include KIT and these genes (shown in bold) in murine and human data are each indicated in red, and form the basis of the specificity filters used to identify novel Kit-interacting receptors (see Results).

FIG. 3. Stimulation of Kit results in rapid activation of IL-4R. Cultured M07e cells were stimulated with Kit ligand (KL) for the indicated lengths of time. Activation of IL-4R in these cells was measured by immunoprecipitation of the IL-4R subunits, IL-4Rα (a) and γc (b), followed by immunoblotting for phosphotyrosine (pY, top panels) or IL-4Rα and γc as controls (bottom panels). Both IL-4Rα and γc are phosphorylated within 5 minutes of KL stimulation, indicating that Kit signaling activates IL-4R.

FIG. 4. IL-4R is expressed on the surface of HSC. (a) HSC from murine bone marrow were identified by flow cytometry on the basis of their Lin⁻Sca-1⁺Kit⁺ (LSK) surface phenotype. Antibody-labeled cells were gated as shown (scatter plots, grey polygons) based on forward and side scatter areas (FSC-A and SSC-A), absence of lineage markers and high expression of Sca-1 and Kit. The distribution of fluorescent intensities of IL-4Rα staining of LSK cells (histogram), compared to staining with an isotype control antibody, indicates that murine HSC express IL-4Rα on their surface. (b) Human bone marrow samples were analyzed by FACS to isolate HSC (Lin⁻CD34^{hi}CD38⁻) and two Lin-control populations, CD34+CD38+ and CD34−CD38+ (top left panels). Two CD19⁺ B-cell populations, IL-4Rα⁺ and IL-4Rα⁻ (bottom left panels), were used as positive and negative controls respectively, for IL-4Rα expression. The levels of IL-4Rα mRNA in these cells was measured using RT-PCR and normalized to those of the housekeeping gene GAPDH (right panel). Unlike CD34⁺CD38⁺ and CD34⁻CD38⁺ cells, human HSC (CD34^{hi}CD38⁻) express the IL-4Rα mRNA at ˜60% of the levels in CD19+ cells that express IL-4Rα. (c) IL-4Rα staining of human marrow HSC (Lin⁻CD34^{hi}CD38⁻) suggests that at least a subset of these cells express IL-4Rα on their surface. (d) Phospho-Flow analysis of murine HSC (Lin⁻Sca-1⁺Kit⁺) showing STATE phosphorylation in response to IL-4 stimulation, indicates that IL-4R expressed in HSC is functional.

FIG. 5. IL-4R and Kit are co-expressed on the surface of human HSC. (a) Peripheral blood stem cells (PBSC) from G-CSF-primed human donors were analyzed by flow cytometry to identify HSC (Lin⁻CD34^{hi}CD38⁻) and a non-HSC control population (Lin⁻CD34⁻CD38⁺). (b) Analysis of Kit staining (compared to isotype control staining) indicates that these cells are Kit⁺. (c, d) Analysis of IL-4Rα staining suggests that at least a subset of HSC express IL-4Rα on their surface (c), whereas none of the CD34⁻CD38⁺ non-HSC do so (d). (e) IL-4Rα staining of M07e cells, known to be IL-4Rα⁺, is shown for comparison.

FIG. 6. 5 male and 5 female Balb/c mice were compared with 5 male and 5 female Balb/c mice with the IL-4 receptor knocked out. While changes were seen in many hematological tests and measurements (listed in column 1), hemoglobin (HGB), percent and absolute Neutrophil concentration, absolute lymphocyte concentration, absolute monocyte concentration, absolute eosinophil concentration, and absolute platelet concentration were statistically significantly different after knockout of the IL-4 receptor. The numbers indicated in color are statistically different (P<0.05) between control and IL-4R animals of the same sex. Other abbreviations: WBC: white blood cell count, RBC: red blood cell count, HCT: hematocrit, MCV: mean corpuscular volume, MCH: mean corpuscular hemoglobin, MCHC: mean corpuscular hemoglobin concentration, Abs: absolute concentration.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Methods and compositions are provided for modulating Kit/stem cell factor receptor (SCFR)/CD117 and interleukin 4 receptor (IL-4R) signaling in a cell in vitro and in vivo, and for identifying candidate agents with activity in modulating Kit and IL-4R signaling. These methods find particular use in treating disorders of the hematopoietic system and in modulating hematopoietic stem cell expansion. These and other objects, advantages, and features of the invention will become apparent to those persons skilled in the art upon reading the details of the compositions and methods as more fully described below.

“Kit”, “Kit receptor”, “stem cell factor receptor”, “SCFR”, and “CD117” are used interchangeably herein to refer to the protein is a type 3 transmembrane receptor for kit ligand (also known as MGF (mast cell growth factor) and as stem cell factor (SCF)). The amino acid sequence for full-length Kit and the nucleic acid sequence may be found at Genbank Accession Nos. NM_{—}000222 (isoform 1) (SEQ ID NO:1, SEQ ID NO:2) and NM_{—}001093772 (isoform 2) (SEQ ID NO:3, SEQ ID NO:4). Constitutively activating mutations in the Kit gene are associated with gastrointestinal stromal tumors, mast cell disease including systemic mastocytosis, and acute myelogenous leukemia. Loss of Kit function is associated with piebaldism.

“Kit modulating agents” is used herein to refer to agents that modulate Kit signaling, i.e. agents that activate/promote/enhance Kit signaling (“Kit activators”) and agents that reduce/suppress/inhibit/antagonize Kit signaling (“Kit inhibitors”). By “an effective amount of a Kit modulating agent”, it is meant an amount of agent that is effective in modulating Kit signaling by about 1.5 fold or more, i.e. 1.5-fold, 2-fold, 3-fold, 4-fold, 5-fold, 7-fold, 10-fold, 15-fold, or 20-fold or more. For example, an effective amount of Kit activator is the effective amount of agent to activate, promote, or enhance Kit signaling such that Kit signaling increases by about 1.5 fold or more, e.g. 1.5-fold, 2-fold, 3-fold, 4-fold, 5-fold, 7-fold, 10-fold, 15-fold, or 20-fold or more relative to Kit signaling in the absence of the agent. Similarly, an effective amount of Kit inhibitor is the effective amount of agent to reduce, suppress, or inhibit Kit signaling by about 1.5 fold or more, e.g. 1.5-fold, 2-fold, 3-fold, 4-fold, 5-fold, 7-fold, 10-fold, 15-fold, or 20-fold or more relative to Kit signaling in the absence of the agent. The modulation of Kit signaling and the efficacy of agents in modulating Kit signaling may be assessed and measured by any convenient method known in the art. For example, the extent of tyrosine phosphorylation on Kit can be assessed by Western blotting with phosphotyrosine specific antibodies, e.g. 4G10 (Upstate, Lake Placid N.Y.), where increased phosphorylation is indicative of increased activity. Alternatively or additionally, the activation state of signaling molecules that are known downstream targets of Kit activity may be assessed, e.g. by measuring the phosphorylation of for example Erk1/2, c-jun N-terminal kinase, PI3 kinase, and/or Akt, as described in, e.g. Hong, L. et al. (2004) Molecular and Cellular Biology 24(3):1401-1410, the disclosure of which is incorporated herein by reference.

“Interleukin 4 receptor”, “IL-4R”, “IL4R”, and “CD124” are used herein to refer to the type 1 transmembrane receptor that can bind interleukin 4 (IL-4) to regulate IgE production. IL-4R is formed by the dimerization of the IL4R alpha subunit (IL4Rα) with the Interleukin Receptor common gamma chain (IL2RG, also known as the γc subunit, or CD132). Membrane-bound IL4Rα is encoded by exons 3 to 7 (extracellular domain), exon 9 (transmembrane domain), and exons 10 to 12 (intracellular domain). A soluble form of IL-4Rα can be produced by proteolysis of the membrane-bound protein, or by alternative splicing that retains exons 3 to 8 while splicing out the exons for the transmembrane (exon 9) and intracellular (exons 10-12) regions. Soluble forms of IL4R can inhibit IL4-mediated activity. The amino acid sequence for the full length IL-4Rα and the nucleic acid sequence that encodes it may be found at Genbank Accession Nos. NM_{—}000418 (isoform a, the membrane bound form) (SEQ ID NO:5, SEQ ID NO:6) and NM_{—}001008699 (isoform b, the soluble form of the receptor) (SEQ ID NO:7, SEQ ID NO:8). The amino acid sequence for the full length γc polypeptide and the nucleic acid sequence that encodes it may be found at Genbank Accession No. NM_{—}000206 (SEQ ID NO:9, SEQ ID NO:10).

“IL-4R modulating agents” is used herein to refer to agents that modulate IL-4R signaling, i.e. agents that activate/promote/enhance IL-4R signaling (“IL-4R activators”) and agents that reduce/suppress/inhibit/antagonize IL-4R signaling (“IL-4R inhibitors”). By “an effective amount of an IL-4R modulating agent”, it is meant an amount of agent that is effective in modulating IL-4R signaling by about 1.5 fold or more, i.e. 1.5-fold, 2-fold, 3-fold, 4-fold, 5-fold, 7-fold, 10-fold, 15-fold, or 20-fold or more. For example, an effective amount of an IL-4R activator is the effective amount of agent to activate, promote, or enhance IL-4R signaling such that IL-4R signaling increases by about 1.5 fold or more, e.g. 1.5-fold, 2-fold, 3-fold, 4-fold, 5-fold, 7-fold, 10-fold, 15-fold, or 20-fold or more relative to IL-4R signaling in the absence of the agent. Similarly, an effective amount of an IL-4R inhibitor is the effective amount of agent to reduce, suppress, or inhibit IL-4R signaling by about 1.5 fold or more, e.g. 1.5-fold, 2-fold, 3-fold, 4-fold, 5-fold, 7-fold, 10-fold, 15-fold, or 20-fold or more relative to IL-4R signaling in the absence of the agent. The modulation of IL-4R signaling and the efficacy of agents in modulating IL-4R signaling may be assessed and measured by any convenient method known in the art. For example, the extent of tyrosine phosphorylation on IL-4Rα can be assessed, e.g. by Western blot hybridization with anti-tyrosine antibody 4G10, where an increase in phosphotyrosines is indicative of an increase in IL-4R activity. Alternatively or additionally, the activation state of signaling molecules that are known downstream targets of IL-4R activity may be assessed, e.g. by assessing the phosphorylation state of Ser473 and/or Thr308 in IRS-2/PI-3K/protein kinase B (PKB) with anti-pSer473-PKB and anti-pThr308-PKB antibodies, or assessing for the presence of the phosphorylated form of Jak1, i.e., activated Jak1, with antiphospho-Jak1 antibodies. Alternatively or additionally, the activity of transcription factors downstream of IL-4R signaling may be assessed, e.g. STATE, NF-κB, etc., by assaying for nuclear localization of the transcription factor by, e.g., EMSA or immunohistochemistry, or the transcription of target genes by, e.g., RT-PCR.

The term “Kit+IL-4R+” cells is used herein to mean cells that express Kit protein and IL-4Rα protein on their surface. It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”. The staining intensity of cells can be monitored by flow cytometry, where lasers detect the quantitative levels of fluorochrome (which is proportional to the amount of cell surface marker bound by specific reagents, e.g. antibodies). Although the absolute level of staining may differ with a particular fluorochrome and reagent preparation, the data can be normalized to a control. In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. “Low” positively stained cells have a level of staining that is above the brightness of an isotype matched control, but is not as intense as the most brightly staining cells normally found in the population. An alternative control may utilize a substrate having a defined density of marker on its surface, for example a fabricated bead or cell line, which provides the positive control for intensity. Flow cytometry-based techniques can be employed with Kit-specific antibodies and IL-4R specific antibodies to confirm the presence of Kit+IL-4R+ cells in a cell population. Other techniques may also be employed, e.g. immunohistochemistry, western blotting, etc.

The term “stem cell” is used herein to refer to a cell or a population of cells which: (a) is self-renewing, and (b) has the potential to give rise to diverse differentiated cell types. Frequently, a stem cell has the potential to give rise to multiple lineages of cells. As used herein, a stem cell may be a pluripotent stem cell, e.g. an embryonic stem (ES) cell, embryonic germ (EG) cell, or an induced pluripotent stem (iPS) cell, which gives rise to all of embryonic tissues of an organism, i.e. endoderm, mesoderm, and ectoderm lineages; a multipotent stem cell, e.g. a mesenchymal stem cell, which gives rise to at least two of the embryonic tissues of an organism, i.e. at least two of endoderm, mesoderm and ectoderm lineages, or a tissue-specific stem cell, which gives rise to multiple types of differentiated cells of a particular tissue. Tissue-specific stem cells include tissue-specific embryonic cells, which give rise to the cells of a particular tissue, and somatic stem cells, which reside in adult tissues and can give rise to the cells of that tissue, e.g. neural stem cells, which give rise to all of the cells of the central nervous system, satellite cells, which give rise to skeletal muscle, and hematopoietic stem cells, which give rise to all of the cells of the hematopoietic system.

The term “differentiated somatic cell” encompasses any cell in an organism that cannot give rise to all types of cells in an organism. In other words, differentiated somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By an “enriched population of cells” it is meant a population of cells that is substantially comprised of a particular cell of interest. In an enriched population, 50% or more of the cells in the population are the cells of interest, e.g. 50%, 60%, 70%, usually 80%, 85%, 90%, more usually 92%, 95%, or 98%, sometimes as much as 100% of the cells in the population. The separation of cells of interest from a complex mixture or heterogeneous culture of cells may be performed by any convenient means known in the art, for example, by affinity separation techniques such as magnetic separation using magnetic beads coated with an affinity reagent, affinity chromatography, or “panning” with an affinity reagent attached to a solid matrix, eg. plate, or other convenient technique. Other techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the desired cells. The affinity reagents may be antibodies that are specific for Kit and IL-4R. Alternatively, specific receptors or ligands for Kit and IL-4R may be used; peptide ligands and receptor; effector and receptor molecules, T-cell receptors specific for Kit and IL-4R, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. The affinity reagents are added to a suspension of cells, and incubated for a period of time sufficient to bind the available cell surface antigens. The incubation will usually be at least about 5 minutes and usually less than about 60 minutes. It is desirable to have a sufficient concentration of affinity reagent in the reaction mixture, such that the efficiency of the separation is not limited by lack of reagent. The appropriate concentration is determined by titration. The medium in which the cells are separated will be any medium that maintains the viability of the cells, for example, phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

In methods of the invention, Kit+IL-4R+ cells are contacted with IL-4 modulating agents and/or Kit modulating agents under conditions that promote cell survival. As discussed above, Kit+IL-4R+ cells are cells that express Kit and IL-4R on their cell surface. Kit+IL-4R+ cells suitable for use in the method may be in or from any mammalian species, e.g. human, primate, equine, bovine, porcine, canine, feline, etc. They may be contacted with the agents in vivo, i.e. in the individual. In such cases, the cells are typically part of a heterogeneous population of cells, e.g. a heterogeneous population of stem cells, undifferentiated somatic cells, differentiated somatic cells, etc., for example, as part of a tissue, e.g. bone marrow, or a body fluid, e.g. peripheral blood. Alternatively, the Kit+IL-4R+ cells may be contacted with the modulating agents in vitro, i.e. in cell culture, either as an enriched population of Kit+IL-4R+ cells or as part of a heterogeneous population of cells, e.g. a heterogeneous population of stem cells, a heterogeneous population of stem cells and somatic cells, a heterogeneous population of stem cells, somatic cells, and cancer cells, etc.

Cells contacted in vivo may be cells that reside in any tissue or body fluid known in the art to comprise Kit+IL-4R+ cells, including, without limitation, bone marrow, peripheral blood, umbilical cord blood, thyroid, secretory gland, brain, retina, skin, gastrointestinal tract, liver, lung, spleen and thymus. Cells contacted in vitro may be cells from a cell line, e.g. a stem cell line or cancer cell line, or they may be primary cells, i.e. cells obtained from an individual. When obtained from an individual, they may be from a neonate, a juvenile or an adult, and from any tissue or body fluid known in the art to comprise Kit+IL-4R+ cells as discussed above. The tissue may be obtained by biopsy or apheresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. In particular in vitro embodiments, the cells are cells of the hematopoietic lineage that have been obtained from a peripheral blood sample or bone marrow sample of an adult donor.

Agents that modulate IL-4R signaling or Kit signaling that may be used to contact the Kit+IL-4+ cells in the present invention include peptides, nucleic acids, and small molecule compounds. For example, agents suitable for modulating IL-4R signaling or Kit signaling in the present invention include nucleic acids, for example, nucleic acids that encode siRNA, shRNA or antisense molecules, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells. Methods for contacting cells with nucleic acid vectors, such as electroporation, calcium chloride transfection, and lipofection, are well known in the art.

Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells may comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Agents suitable for modulating IL-4R signaling or Kit signaling in the present invention also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the polypeptide agent is to modulating signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the polypeptide agent is to modulating signaling extracellularly, the polypeptide may be formulated for improved stability. For example, the peptides may be PEGylated, where the polyethyleneoxy group provides for enhanced lifetime in the blood stream. The SHBG polypeptide may be fused to another polypeptide to provide for added functionality, e.g. to increase the in vivo stability. Generally such fusion partners are a stable plasma protein, which may, for example, extend the in vivo plasma half-life of the SHBG polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the SHBG polypeptide also will be produced and employed as a multimer having substantially the same structure as the stable plasma protein precursor. These multimers will be homogeneous with respect to the polypeptide agent they comprise, or they may contain more than one polypeptide agent.

Stable plasma proteins are proteins which typically exhibit in their native environment an extended half-life in the circulation, i.e. greater than about 20 hours. Examples of suitable stable plasma proteins are immunoglobulins, albumin, lipoproteins, apolipoproteins and transferrin. The polypeptide agent typically is fused to the plasma protein, e.g. IgG at the N-terminus of the plasma protein or fragment thereof which is capable of conferring an extended half-life upon the SHBG polypeptide. Increases of greater than about 100% on the plasma half-life of the SHBG polypeptide are satisfactory. Ordinarily, the SHBG polypeptide is fused C-terminally to the N-terminus of the constant region of immunoglobulins in place of the variable region(s) thereof, however N-terminal fusions may also find use. Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods.

The site at which the fusion is made may be selected in order to optimize the biological activity, secretion or binding characteristics of the SHBG polypeptide. The optimal site will be determined by routine experimentation.

In some embodiments the hybrid immunoglobulins are assembled as monomers, or hetero- or homo-multimers, and particularly as dimers or tetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA immunoglobulin, and occasionally IgG immunoglobulin, may also exist in a multimeric form in serum. In the case of multimers, each four chain unit may be the same or different.

The polypeptide agent for use in the subject methods may be produced from eukaryotic produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention as agents that modulate IL-4R signaling or Kit signaling are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise 20% or more by weight of the desired product, sometimes 75% or more by weight, e.g., 95% or more by weight, and for therapeutic purposes, usually at least about 99.5% by weight, in relation to contaminants related to the method of preparation of the product and its purification. Usually, the percentages will be based upon total protein.

Another example of polypeptide agents suitable for modulating IL-4R signaling or Kit signaling are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents suitable for modulating IL-4R signaling or Kit signaling in the present invention also include small molecules. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992). Small molecule compounds can be provided directly to the medium in which the cells are being cultured, for example as a solution in DMSO or other solvent.

As discussed above, an IL-4R modulatory agent may be an agent that promotes IL-4R signaling or an agent that antagonizes IL-4R signaling. Specific examples of peptide agents that promote IL-4R signaling include, without limitation, IL-4 peptides and polypeptides. Examples of peptide agents that antagonize IL-4R signaling include, without limitation, IL-4R-specific antibody, e.g. antibody specific for IL-4Rα or IL-4R dimer; IL-4-specific antibody; and soluble IL-4Rα peptides and polypeptides. Examples of nucleic acid agents that promote IL-4R signaling include, without limitation, nucleic acids encoding IL-4 peptides and polypeptides. Examples of nucleic acid agents that antagonize IL-4R signaling include, without limitation, siRNA or other forms of inhibitory RNA specific for IL-4Rα or IL-4.

As also discussed above, a Kit modulatory agent may be an agent that promotes Kit signaling or an agent that antagonizes Kit signaling. Specific examples of peptide agents that promote Kit signaling include, without limitation, Kit ligand peptides and polypeptides. Examples of peptide agents that antagonize Kit signaling include, without limitation, Kit-specific antibody and Kit ligand-specific antibody. Examples of nucleic acid agents that promote Kit signaling include, without limitation, nucleic acids encoding Kit ligand and Kit ligand peptides. Examples of nucleic acid agents that antagonize Kit signaling include, without limitation, siRNA or other forms of inhibitory RNA specific for Kit. Examples of small molecule agents that antagonize Kit signaling include, without limitation, tyrosine kinase inhibitors such as Imatinib mesylate/STI571/Gleevac™.

In practicing methods of the invention, the Kit+IL-4R+ cells are contacted with an effective amount of an IL-4R modulating agent and/or an effective amount of a Kit+ modulating agent. As discussed above, “an effective amount of an IL-4R modulating agent”, it is meant an amount of agent that is effective in modulating IL-4R signaling. Likewise, by “an effective amount of a Kit modulating agent”, it is meant an amount of agent that is effective in modulating Kit signaling. The calculation of the effective amount or effective dose of modulatory agent to be administered is within the skill of one of ordinary skill in the art, and will be routine to those persons skilled in the art. See, for example, the discussion above with regard to assessing and measuring the efficacy of Kit modulatory agents and IL-4R modulatory agents in modulating Kit or IL-4R activity, respectively. Needless to say, the final amount to be administered will be dependent upon the route of administration and upon the nature of the disorder or condition that is to be treated.

Cells are contacted with agents under conditions that normally promote the survival of the cells. Cells contact in vivo are typically already in an environment that promotes survival. When contacting Kit+IL-4R+ cells in vitro, conditions that promote their survival include, for example, culturing at about 37° C. in nutrient media such as Iscove's modified DMEM or RPMI 1640, supplemented with goat serum, fetal calf serum, or horse serum (about 5-10%), L-glutamine, a thiol, particularly 2-mercaptoethanol, and antibiotics, e.g. penicillin and streptomycin. Culture medium may be liquid or semi-solid, e.g. containing agar, methylcellulose, etc. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells may be contacted with the modulatory agents in vitro by any of a number of well-known methods in the art. For example, polypeptides (including antibodies) or small molecule agents may be provided to the cells in the media in which the cells are being cultured. Nucleic acids agents may be provided to the cells on vectors under conditions that are well known in the art for promoting their uptake, for example electroporation, calcium chloride transfection, and lipofection. Alternatively, nucleic acids encoding the agent may be provided to the cells via a virus, i.e. the cells are contacted with viral particles comprising the nucleic acid agent. Retroviruses, for example, lentiviruses, are particularly suitable to the method of the invention, as they can be used to transfect non-dividing cells (see, for example, Uchida et al. (1998) P.N.A.S. 95(20):11939-44). Commonly used retroviral vectors are “defective”, i.e. unable to produce viral proteins required for productive infection. Rather, replication of the vector requires growth in a packaging cell line.

Likewise, cells may be contacted with a modulatory agents in vivo by any of a number of well-known methods in the art for the administration of peptides, small molecules and nucleic acids to a subject. The modulatory agent can be incorporated into a variety of formulations. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the modulatory agent can be achieved in various ways, including oral, buccal, rectal, parenteral, intraperitoneal, intradermal, transdermal, intracheal, etc., administration. The active agent may be systemic after administration, i.e., it may be distributed throughout the body, or may be localized, e.g. by the use of regional administration, e.g. intraventricular or intrathecal administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

Methods of the invention find use in increasing the number of erythrocytes, neutrophils, monocytes, eosinophils, and platelets and decreasing the number of lymphocytes in a patient. In such methods, an effective amount of an IL-4R modulating agent to reduce IL-4R signaling is administered. Following this method, the number of circulating erythrocytes, neutrophils, monocytes, eosinophils, and platelets is increased, and the number of lymphocytes is decreased. That is, there are about 1.5-fold, about 2-fold, about 3-fold, about 4-fold, about 6-fold, about 8-fold, about 10-fold more erythrocytes, neutrophils, monocytes, eosinophils, or platelets and about 1.5-fold, about 2-fold, about 3-fold, about 4-fold, about 6-fold, about 8-fold, about 10-fold less lymphocytes after execution of the method, e.g. 12 hours, 24 hours, 48 hours, 72 hours, 5 days, 7 days, or 10 days after execution of the method, than before the method was performed. Such methods find particular use in individuals that have low numbers of circulating erythrocytes, neutrophils, monocytes, eosinophils, or platelets or high numbers of lymphocytes, i.e. patients with anemia, neutropenia, monocytopenia, eosinopenia, thrombocytopenia, etc. Such individuals can be readily identified by any of a number of methods known in the art for determining a complete blood count (CBC), e.g. a manual count of a blood smear, an automatic count with an automated analyzer, etc. Likewise, the effectiveness of the method can also be determined using these methods.

Methods of the invention also find use in increasing the number of erythrocytes, neutrophils, monocytes, eosinophils, and platelets and decreasing the number of lymphocytes in a patient. In such methods, an effective amount of an IL-4R modulating agent to promote IL-4R signaling is administered. Following this method, the number of circulating erythrocytes, neutrophils, monocytes, eosinophils, and platelets is decreased, and the number of lymphocytes is increased. That is, there are about 1.5-fold, about 2-fold, about 3-fold, about 4-fold, about 6-fold, about 8-fold, about 10-fold less erythrocytes, neutrophils, monocytes, eosinophils, or platelets and about 1.5-fold, about 2-fold, about 3-fold, about 4-fold, about 6-fold, about 8-fold, about 10-fold more lymphocytes after execution of the method, e.g. 12 hours, 24 hours, 48 hours, 72 hours, 5 days, 7 days, or 10 days after execution of the method, than before the method was performed. Such methods find particular use in individuals that have high numbers of erythrocytes, neutrophils, monocytes, eosinophils, and platelets and low numbers of lymphocytes, i.e. patients with polycythemia, an infection, atopy, lymphocytopenia, etc. Such individuals can be readily identified by any of a number of methods known in the art for determining a complete blood count (CBC), e.g. a manual count of a blood smear, an automatic count with an automated analyzer, etc. Likewise, the effectiveness of the method can also be determined using these methods.

Methods of the invention also find use in reducing the survival, proliferation, and migration of cancer cells. In such methods, Kit+IL-4R+ cells are contacted with an effective amount of an IL-4R inhibitor. As a result, survival, proliferation, and/or migration of the Kit⁺IL-4R⁺ cancer cells is reduced. By reduced, it is meant that about 1.5-fold, about 2-fold, about 3-fold, about 4-fold, about 6-fold or about 8-fold more Kit+IL-4R+ cells die, stop proliferating, and/or stop migrating in the population contacted with IL-4R inhibitor than do in the population not contacted with IL-R inhibitor. As a result, tumor growth may be inhibited at 5% or more, 10% or more, 20% or more, sometimes 20% to 50% or more, in some instances by 50% or more (e.g., 50% to 70%, 80%, 90%, or 100%) as compared to the appropriate control, the control typically being a cancer not treated with the IL-4R inhibitor. Typically, the methods of the invention provide for a reduced survival, proliferation, and/or migration that is at least two-fold or higher than that of the control.

Methods of the invention also find use in enhancing the responsiveness of Kit+IL-4R+ cells to a Kit inhibitor, for example, to reduce the survival, proliferation, and/or migration of cancer cells. In such methods, Kit+IL-4R+ cells are contacted with an effective amount of a Kit inhibitor and an effective amount of an IL-4R inhibitor in a combination therapy. As a result, survival, proliferation, and/or migration of the Kit⁺IL-4R⁺ cancer cells is reduced. This reduction in survival, proliferation and/or migration is more significant, i.e. is “enhanced”, relative to the reduction in survival, proliferation and/or migration of Kit+IL-4R+ cells when Kit inhibitors are provided alone, i.e. in the absence of the IL-4R inhibitor. By “enhanced”, it is meant the response is at least about 150% of the response of the population contacted with Kit inhibitor in the absence of IL-R inhibitor, about 200%, about 300%, about 400%, about 600%, or about 800% of the response of the population contacted with Kit inhibitor in the absence of IL-R inhibitor. In other words, the about 1.5-fold, about 2-fold, about 3-fold, about 4-fold, about 6-fold or about 8-fold more Kit+IL-4R+ cells die, stop proliferating, and/or stop migrating in the population contacted with both Kit inhibitor and IL-4R inhibitor than do in the population contacted with Kit inhibitor in the absence of IL-R inhibitor. As a result, tumor growth may be inhibited 5% or more, 10% or more, 20% or more, sometimes from about 20% to about 50%, in some instances, 50% or more, e.g., 60% or more, 70% or more, 80% or more, 90% or more, or 100% as compared to the appropriate control, e.g. a cancer not treated with the IL-4R inhibitor. In some instances, the methods of the invention provide for an increased responsiveness that is at least about two-fold or higher. Such methods find particular use in treating patients with cancer, e.g. a lymphoma or leukemia.

For inclusion in a medicament, modulatory agents may be obtained from a suitable commercial source. As a general proposition, the total pharmaceutically effective amount of the modulatory agent compound administered parenterally per dose will be in a range that can be measured by a dose response curve.

A modulatory agent to be used for therapeutic administration must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The modulatory agent ordinarily will be stored in unit or multi-dose containers, for example, sealed ampules or vials, as an aqueous solution or as a lyophilized formulation for reconstitution. As an example of a lyophilized formulation, 10 mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations. Other uses include investigations where it is desirable to investigate a specific effect of the modulation of Kit and/or IL-4R signaling.

The methods of the present invention also find use in combined therapies. For example, a number of agents may be useful in the treatment of lymphomas and leukemias, e.g. rituximab, gleevac, etc. The combined use of the Kit and IL-4R modulatory agents of the present invention and these other agents may have the advantages that the required dosages for the individual drugs is lower, and the effect of the different drugs complementary.

The methods of the invention also find several uses in vitro. For example, methods of the invention find use in enhancing or augmenting the proliferation of stem cells in vitro. In such methods, a culture comprising Kit⁺IL-4R⁺ stem cells is contacted with an effective amount of a Kit activator and an effective amount of an IL-4R activator. As a result, proliferation in the culture is increased. This increase in proliferation is more significant, i.e. is enhanced, relative to the increase in proliferation that would be observed of a culture that was contacted with only a Kit activator. By “enhanced”, it is meant there are about 150% or more cells in the culture than would be observed in a culture that was contacted with Kit inhibitor in the absence of IL-R inhibitor, e.g. about 200%, about 300%, about 400%, about 600%, or about 800% more cells in the population contacted with Kit inhibitor in the absence of IL-R inhibitor. In other words, there is about a 1.5-fold, about a 2-fold, about a 3-fold, about a 4-fold, about a 6-fold or about an 8-fold elevation in the number of cells in the culture than what would be observed in a culture contacted with Kit activator in the absence of IL-R activator.

The methods described herein provide a useful system for screening candidate agents for activity in reducing Kit signaling and, hence, in reducing the survival, proliferation, and migration of Kit+IL-4R+ cells in cancer. To that end, it has been shown that agents that reduce IL-4R signaling have a potent effect on reducing Kit signaling. Accordingly, a reduction in IL-4R signaling may be used as a surrogate readout for the reduction of Kit signaling. In other words, Kit signaling can be measured as a function of IL-4R activity.

In screening assays for biologically active agents, Kit+IL-4R+ cells, usually cultures of cells, are contacted with the agent of interest in the presence of an agent, and the effect of the candidate agent is assessed by monitoring output parameters reflective of IL-4R activity, such as the amount of tyrosine phosphorylation on IL-4R, the phosphorylation state of Ser473 or Thr308 on protein kinase B, the presence of the phosphorylated form of Jak1, the activity of transcription factors downstream of IL-4R signaling, e.g. STATE, NE-κB, etc., and the like by methods described above.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For example, western blots or protein arrays may be employed to measure phosphorylation. EMSA gel shifts, immunohistochemistry, or RT-PCR may be employed to measure transcription factor activity. Such methods would be well known to one of ordinary skill in the art.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

There is functional evidence of synergy in vivo between Kit and Type I cytokine receptor pathways (Duarte, R. F. & Franf, D. A. Leuk Lymphoma 43, 1179-1187 (2002); Sui, X. et al. Blood 92, 1142-1149 (1998)). Biochemical data suggests that the mechanism for synergy involves direct interaction of the Type I cytokine receptors EpoR and IL-7R with activated Kit (Jahn, T. et al. Blood 110, 1840-1847 (2007) Wu, H. et al. Nature 377, 242-246 (1995)). The interactions between Kit and these Type I cytokine receptors are functionally important, as illustrated by experiments demonstrating synergistic effects of the loss of function of Kit and γc, a subunit of IL-7R (Rodewald, H. R. et al. Immunity 6, 265-272 (1997)). We set out to identify novel interactions between Kit and other receptors in hematopoietic cells. For this purpose, a bioinformatics approach called Co-expressed RNA for Signal Transduction Elucidation, or CORSiTE, was developed that analyzes gene expression data from vast, publicly available repositories across species to identify novel candidate signaling interactions, based on the hypothesis that signaling components co-expressed with target receptors at the RNA level in relevant cell types are ideal candidate proteins for investigation for signal transduction and propagation. Using this approach, IL-4R was newly identified as a Kit-activated receptor in hematopoietic cells.

### Materials and Methods

Generation of the curated gene expression database. Individual GEO Samples (GSMs) were identified by searching NCBI GEO for the occurrence of the search terms (“hematopoietic stem”, “kidney”, “lung” and “adipocyte”) in the GSM annotation fields. GSMs were filtered to remove those that lacked expression measurements for at least 50% of genes. A list of the GSM identifiers can be found in Table 1. Curation of the human hematopoietic GSMs was performed by comparing the methods in the GSM method field to standard procedures for isolation of HSC. GSMs thus identified to represent samples with higher stringency in HSC selection are indicated in bold in Table 1.

TABLE 1
GSM identifiers of NCBI GEO gene expression samples used in this study.

Human hematopoietic progenitors
(samples with higher stringency in selection for HSC are indicated in bold)
2840, 86779, 86781, 86783, 87693, 87695, 87697, 87699, 87701, 87703, 87705, 87707, 87709,
87711, 87713, 87715, 87717, 87719, 87721, 87723, 87725, 87727, 87729, 87731, 87733, 88002,
88003, 88004, 88005, 88006, 88007, 88008, 88009, 88010, 88011, 88012, 88013, 88014, 88015,
88016, 88017, 88018, 88019, 88020, 88021, 88022, 88023, 88024, 88025, 88026, 88027, 88028,
88029, 88030, 88031, 88032, 88033, 88034, 88035, 88036, 88037, 112271, 112272, 112273, 112274,
112275, 112276, 112277, 112278, 112279, 112280, 112281, 112282, 112283, 112284, 112285,
112286, 112287, 112288, 112289, 112290, 112291, 112292, 112293, 112294, 112295, 112296,
112297, 112298, 116315, 116316, 116317, 116318, 116323, 116324, 116325, 116326, 137433,
137434, 137435, 137436, 137437, 137438, 137439, 137440, 137441, 137442, 137443, 137444,
137445, 137446, 137447, 137448, 137449, 137450, 137451, 137452, 137453, 137454, 137455,
137456, 137457, 137458, 137459, 137460, 137461, 137462, 137463, 137464, 137465, 137466,
137467, 137468, 137469, 137470, 137471, 137472, 137473, 137474, 137475, 137476, 137477,
137478, 137479, 137480, 137481, 137482, 137483, 137484, 137485, 137486, 137487, 137488,
137489, 137490
Human lung
179572, 179584, 179585, 180038, 180039, 180040, 180041, 180042, 180043, 180058, 183436,
183437, 183438, 183439, 191013, 194462, 194463, 194464, 198616, 198617, 198618, 198619,
198620, 198621, 198622, 198623, 198624, 198625, 198626, 198627, 198628, 198629, 198630,
198631, 198632, 198633, 198634, 198635, 198636, 198637, 198638, 198639, 198640, 198641,
198642, 198643, 198644, 198645, 198646, 198647, 198648, 198649, 198650, 198651, 198652,
198653, 198654, 198655, 198656, 198657, 198658, 198659, 198660, 198661, 198662, 198663,
198664, 198665
Human kidney
133550, 133561, 133563, 133573, 133574, 133577, 133582, 133584, 133587, 133593, 133594,
133615, 133626, 133630, 133638, 133657, 144420, 144421, 144422, 144423, 144425, 144427,
162148, 162149, 162150, 162151, 162152, 162153, 162154, 162155, 162156, 162157, 162158,
162159, 162160, 162161, 162162, 162163, 162164, 162165, 162166, 162167, 162168, 162169,
162170, 162171, 162172, 162173, 162174, 162175, 162176, 162177, 162178, 162179, 162180,
162181, 162182, 162183, 162184, 162185, 162186, 162187, 162188, 162189, 162190, 162191,
162192, 162193, 162194, 175911, 176321, 176322, 176323, 176324, 176424, 176425, 176426,
176427, 178455, 178456, 178457, 178458, 178459, 178460, 178461, 178463, 178464, 178465,
178466, 178467, 178468, 178469, 178470, 178471, 178472, 178473, 178498, 178500, 178501,
178502, 178503, 178504, 178506, 178507, 178508, 178509, 178510, 178511, 183307, 183308,
183309, 183310, 183311, 183312, 183313, 183314, 194519, 194520, 194521, 194522, 194523,
194524, 198783, 198785
Human adipocyte
18975, 18976, 28475, 28476, 28477, 28478, 28479, 28480, 28481, 28482, 28483, 28484, 28485,
28486, 28487, 28488, 28489, 28490, 28491, 28492, 28493, 28494, 28495, 28496, 28497, 28498,
30645, 30646, 30647, 30648, 30649, 30650, 30651, 30652, 30653, 30654, 30655, 30656, 30657,
30658, 30659, 30660, 30661, 30662, 47224, 47225, 47226, 47227, 47228, 47229, 47230, 47231,
47232, 47233, 47234, 47235, 47242, 47256, 47269, 47286, 47299, 47300, 47301, 47303, 47317,
47319, 47321, 47322, 47323, 47324, 47325, 47326, 47327, 47328, 47329, 47330, 47331, 47332,
47333, 47334, 47335, 47336, 47337, 47878, 47879, 47880, 47881, 47882, 47883, 47884, 47885,
47886, 47887, 47888, 47889, 47890, 47891, 47892, 47893, 47894, 47895, 47896, 47897, 47898,
47899, 47900, 47901, 47902, 47903, 47904, 47905, 47906, 47907, 47908, 47909, 47910, 47911,
47912, 47913, 47914, 47935, 47936, 47937, 47938, 47939, 47940, 47941, 47942, 47943, 47944,
47945, 47946, 47947, 47948, 47950, 47951, 47952, 47953, 47954
Murine hematopoietic progenitors
36673, 36674, 36675, 36676, 36677, 36678, 36679, 36680, 36681, 36682, 36683, 36684, 36685,
36686, 36687, 36688, 36689, 36690, 36691, 36692, 36693, 36694, 36695, 36696, 36697, 36698,
36699, 36700, 36701, 36702, 36703, 36704, 36705, 36706, 36707, 36708, 36709, 36710, 36711,
36712, 36713, 36714, 36715, 36716, 72879, 72881, 72883, 72899, 72901, 72903, 72905, 72907,
72909, 99337, 99338, 132914, 132915, 149526, 149527, 149528, 149529, 149530, 149531, 149532,
149533, 149536, 149537, 149538, 149539, 153707, 153709, 153710, 153712, 153714, 153716,
153717, 153718, 153719, 153720, 153721, 153722
Murine lung
34855, 34858, 34859, 34860, 34869, 34870, 34871, 34872, 187404, 187405, 187406, 187407,
187408, 187409, 187410, 187411, 187412, 187413, 187414, 187415, 187416, 187417, 189472,
189473, 189474, 189475, 189476, 189477, 189478, 189479, 189791, 189792, 189793, 189794,
189795, 189796, 189797, 189798, 189799
Murine kidney
34855, 34858, 34859, 34860, 34869, 34870, 34871, 34872, 141682, 142097, 142098, 142099,
142100, 142101, 142102, 144585, 144586, 144587, 144588, 144589, 144590, 144591, 144592,
144593, 144594, 144595, 144596, 144597, 144598, 144599, 144600, 144601, 144602, 144603,
144604, 144605, 144606, 144607, 144608, 144609, 144610, 144611, 144612, 144613, 144614,
144615, 144616, 144617, 144618, 144619, 144620, 144621, 152245, 152246, 152247, 152248,
152249, 152250, 152251, 152252, 152253, 152254, 152255, 154540, 154541, 154542, 154543,
154544, 154545, 154546, 154547, 154548, 154549, 155089, 155090, 155091, 155100, 155101,
155102, 159983, 159984, 159985, 159986, 174757, 174758, 174759, 174760, 174761, 174762,
174763, 174764, 174891, 203834, 203835, 203836
Murine adipocyte
38277, 38278, 38279, 38280, 38281, 38282, 38283, 38284, 38285, 38286, 38287, 38288, 38289,
38290, 38291, 38292, 38293, 38294, 39234, 39235, 39236, 39237, 39238, 39239, 39240, 39241,
39242, 39243, 39244, 39245, 39246, 39247, 39248, 39249, 39250, 39251, 39252, 39253, 39254,
39255, 39256, 39257, 39258, 39259, 39260, 39261, 39262, 39263, 39264, 39265, 39266, 39267,
39268, 39269, 39270, 39271, 39272, 39273, 39274, 39275, 39276, 39277, 39278, 39279, 39280,
39281, 39282, 39283, 39284, 39285, 39286, 48670, 48673, 48674, 48675, 48676, 48677, 48678,
48679, 48680, 48862, 48893, 48894, 48895, 48896, 48897, 48898, 48899, 48900, 48901, 48902,
48903, 48904, 48905, 48906, 48907, 48908, 48909, 48910, 162532, 162533, 162534, 162535,
162536, 162537, 162538, 162539, 162540, 162541, 162542, 162543, 162544, 162545, 162546,
162547, 162548, 162549, 162550, 162551, 162552, 162553, 162554, 162555

Analysis of gene expression data. Gene expression measurements were mapped from platform-specific probes/probesets to Entrez GeneIDs using AILUN (Chen, R. et al. Nat Methods 4, 879, 1107-879 (2007)). In cases where multiple probesets mapped to a single GeneID, the expression of these probesets was averaged. The expression data from each GSM were then rank-normalized to allow for comparisons between different microarray platforms, as previously described and validated (Dudley, J. T. et al. Mol Syst Biol 5, 307 (2009)). We restricted our analysis to Kit and the 25 human or 24 mouse genes encoding the candidate Type I cytokine receptors (Table 2, below) and the GSMs of interest for each organism (Table 1). These genes were clustered hierarchically based on their expression profile across hematopoietic progenitors (HP) and control tissues (lung, kidney, adipocyte) on the basis of the commonly used Euclidean distance measure and complete linkage agglomerative clustering method.

Cell culture. M07e cells (DSMZ, Braunschweig, Germany) were cultured in RPMI-1640 (Invitrogen/Gibco, Carlsbad, Calif.) with 10% fetal calf serum (FCS; Omega Scientific, Tarzana, Calif.) in the presence of human IL-3 (20 ng/ml; Biosource, Camarillo, Calif.) and human IL-4 (20 ng/ml; R&D Systems Inc, Minneapolis, Minn.). For stimulation of Kit with Kit ligand (KL), M07e cells were growth factor—as well as serum-deprived for 12 hours prior to stimulation with 250 ng/ml of recombinant human KL (Biosource, Camarillo, Calif.).

Immunoprecipitation and immunoblotting. Immunoprecipitation and immunoblotting were performed as reported previously (Mani, M. et al. Blood 114, 2900-2908 (2009)). Following stimulation with KL, cells were collected in ice-cold PBS containing 1 mM sodium ortho-vanadate (Sigma, St. Louis, Mo.), resuspended at a density of 1×10⁷-2×10⁷ cells/ml in lysis buffer containing 10 mM Tris-HCl (pH7.4), 150 mM NaCl, 20 mM sodium phosphate (pH7.4), 10 mM sodium pyrophosphate (pH7.4), 5 mM EDTA, 1 mM sodium ortho-vanadate, 1 mM glycerophosphate (Sigma) and 1% Triton X-100, and lysed. Protease inhibitors (Complete Protease Inhibitor Cocktail, Roche, Indianapolis, Ind.) were added according to manufacturer's recommendations. Post-nuclear supernatants were subjected to one round of pre-clearing with Protein A Sepharose (Amersham/Pharmacia, Piscataway, N.J.). 3-6 μg of anti-human IL-4Rα (C-20; Santa Cruz Biotechnology, Santa Cruz, Calif.) or anti-human γc (clones N-20 and C-20, Santa Cruz Biotechnology) antibody was used per IP, and antibody-protein complexes were collected with 50 μl to 70 μl of Protein A Sepharose. Western blotting was performed as previously described (Wu, H. et al. Nature 377, 242-246 (1995); Mani, M. et al. Blood 114, 2900-2908 (2009)) using anti-phosphotyrosine antibody clones 4G10 (Upstate, Lake Placid, N.Y.) and PY20 (BD Biosciences, San Jose, Calif.).

Flow cytometry and FACS analysis. To analyze surface expression of IL-4Rα in murine Lin-Sca-1+Kit+ HSC, bone marrow cells were prepared by flushing the femur and tibia of 4 to 8 week old mice (C57/B6) with cold RPMI-1640 containing 2% FBS. Following depletion of red blood cells (RBC) using red cell lysis buffer (eBioscience, San Diego, Calif.), cells were washed and resuspended in cold RPMI-1640 containing 2% FBS. Approximately 10⁷ cells were pre-treated with anti-mouse CD16/CD32 antibody (clone 2.4G2, BD Biosciences) for 10 min to block Fc receptors and then stained with APC-conjugated rat anti-mouse CD117/Kit (clone 2B8, BD Biosciences), PE-Cy5.5-conjugated rat anti-mouse Ly-6A/E/Sca-1 (clone D7, Invitrogen Corp., Camarillo, Calif.), and Alexa Fluor 488-conjugated Lineage mixture comprising antibodies against mouse CD3e, CD11b, CD45R, Ly-6C/G and TER-119 (Invitrogen). IL-4Rα was labeled using PE-conjugated rat anti-mouse CD124/IL-4Rα (clone MIL4R-M1, BD Biosciences), while PE-conjugated rat anti-mouse IgG2a,κ (BD Biosciences) was used as the isotype control.

Expression of IL-4Rα in human Lin-CD34+CD38-HSC was measured in samples of G-CSF-mobilized peripheral blood stem cells (G-PBSC), obtained in accordance with a protocol approved by the Stanford University Institutional Review Board (IRB). The cells were resuspended in RPMI-1640, depleted of dead cells and RBC by density centrifugation (Histopaque-1083, Sigma-Aldrich, St Louis, Mo.), then washed and resuspended in cold PBS containing 2% FBS. The cells were pre-treated with FcR blocking reagent (human IgG, Miltenyi Biotech Inc, Auburn, Calif.) for 10 min, then treated with CD34 microbeads for 30 min after which positive cells were selected using Miltenyi AutoMACS (Miltenyi Biotech Inc). Cells were then stained with various combinations of the following as indicated in the figure legends: APC-conjugated mouse anti-human CD38 (clone HB7, BD Biosciences); PE-Cy7-conjugated mouse anti-human CD34 (clone 8G12, BD Biosciences); FITC-conjugated Lineage mixture comprising antibodies against human CD3, CD14, CD19, CD20 and CD56 (BD Biosciences); FITC-conjugated mouse anti-human CD19 (clone HIB19, BD Biosciences); and either PE-conjugated mouse anti-human CD124/IL-4Rα (clone hIL4R-M57, BD Biosciences) or PE-conjugated mouse IgG1κ (clone MOPC-21, BD Biosciences) as the isotype control.

For analysis of surface expression of IL-4Rα in M07e cells, cultured cells were serum- and growth factor-deprived for 12 hours, then stained with PE-conjugated mouse anti-human CD124 to label IL-4Rα or PE-conjugated mouse IgG1k as isotype control and analyzed using a FACSCalibur flow cytometer (BD Biosciences).

Flow cytometry and fluorescence activated cell sorting (FACS) of antibody labeled cells was performed using a FACS ARIA II (BD Biosciences). Data were analyzed using FlowJo software.

Real-time PCR analysis of FACS-sorted cells. FACS-sorted cell populations were collected in RNAProtect Cell reagent (Qiagen Inc, Valencia, Calif.) prior to RNA extraction using the RNeasy Plus Micro Kit (Qiagen Inc). RNA was reverse-transcribed using Superscript III reverse transcriptase and random primers (Invitrogen) according to the manufacturer's instructions. Analysis of gene expression was carried out by TaqMan real-time PCR, using primers and probes for GAPDH (Hs99999905) and IL4RA (Hs00166237) from Applied Biosystems, Foster City, Calif.

Intracellular phospho-STAT6 analysis. Cells derived from c-Kit(BAC)-EGFP mice that are transgenic for a bacterial artificial chromosome expressing EGFP under the control of the Kit promoter were used instead of anti-Kit antibodies (Tallini, Y. N. et al. Proc Natl Acad Sci USA 106, 1808-1813 (2009)). Freshly isolated murine bone marrow mononuclear cells were suspended in cold RPMI-1640 containing 10% FBS and stained with PE-CY7 conjugated anti-mouse Sca-1 (BD Biosciences, San Jose, Calif., Cat 558162) and PE conjugated anti-mouse lineage cocktail (Biolegend, San Diego, Calif. Cat: 78035) at 4° C. for 60 min. The cells were washed with cold RPMI-1640 containing 10% FBS and resuspended in pre-warmed RPMI-1640 containing 10% FBS at concentration of 5-10 million cells/ml. Cells were transferred as 0.5 ml aliquots into flow cytometry test tubes (BD Biosciences, Falcon 2052), rested at 37° C. for 1 hr and stimulated with 1 ng/ml of recombinant mouse IL-4 (Invitrogen, Cat: PMC 0045) for 15 min. Cells were fixed by adding 25 μl of 32% paraformaldehyde (EM grade, Electron Microscopy Sciences, Hatfield, Pa., Cat: 15714) at room temperature for 10 min. Cells were washed (centrifugation at 1800 rpm for 5 min) with staining buffer (phosphate-buffered saline with 2% FBS) and permeabilized by resuspension in 1 ml ice-cold 95% methanol (HPLC grade, Fischer Scientific, Cat. A452-4) for 10 min. Cells were stored at 20° C. for overnight before staining for flow cytometry. PFA-fixed, methanol-permeabilized cells were rehydrated by adding 4 ml staining buffer and washed (centrifugation at 1800 rpm for 5 min) twice with staining buffer. Cells were resuspended in 100 μl staining buffer and stained with Alexa Flour-647 conjugated anti-mouse phospho-STAT6 (BD Biosciences, San Jose, Calif., Cat. 558242) at room temperature for 30 min. Samples were washed (centrifugation at 1800 rpm for 5 min) once with staining buffer and analyzed on an FACS Aria II (BD Biosciences). The results were analyzed using Flow Jo software.

### Results

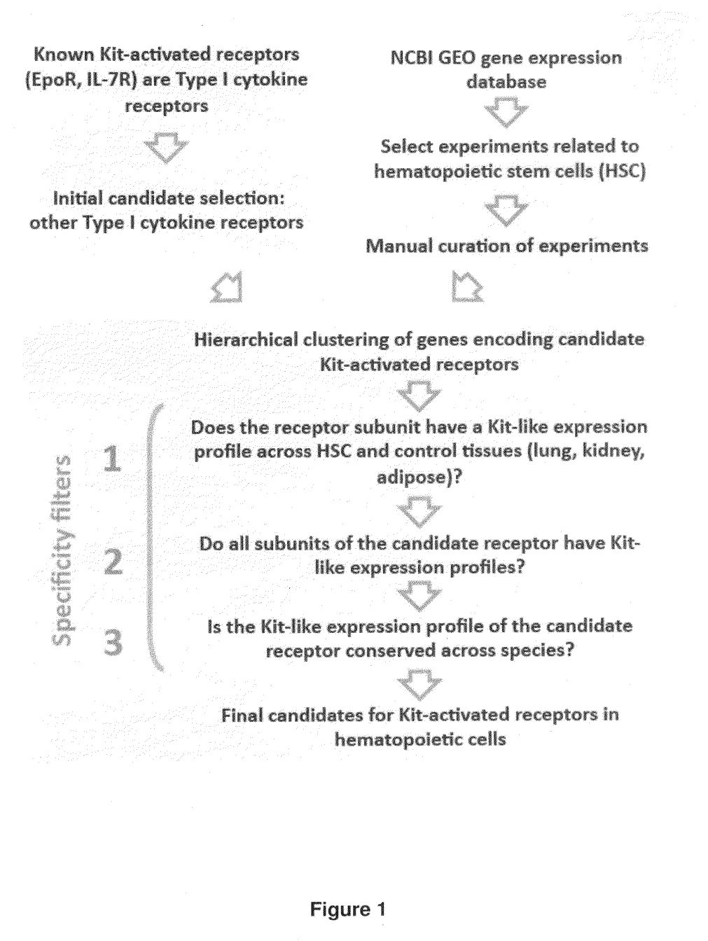

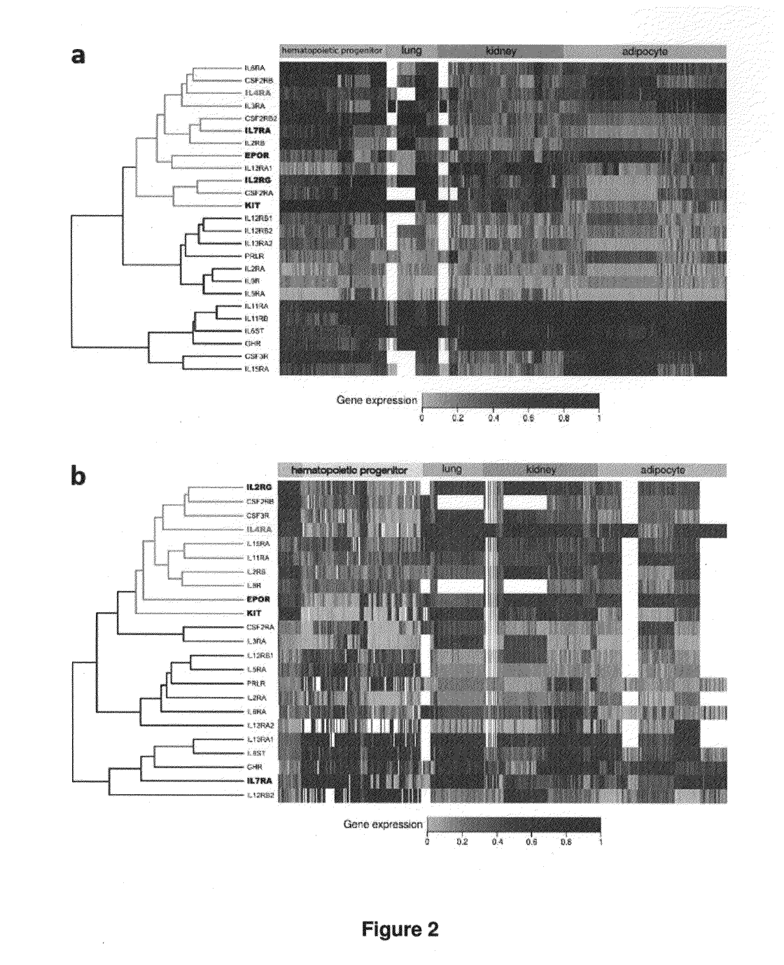

Prediction of novel Kit-activated receptors in hematopoietic cells. A guilt-by-association (GBA) approach was used based on co-expression and comparative biology to predict novel Kit-activated receptors in hematopoietic cells (FIG. 1). Since functionally related genes tend to be co-expressed (Eisen, M. B. et al. Proc Natl Acad Sci USA 95, 14863-14868 (1998); Wolfe, C. J. et al. BMC Bioinformatics 6, 227 (2005); Lee, H. K. et al. Genome Res 14, 1085-1094 (2004); Stuart, J. M. et al. Science 302, 249-255 (2003)), candidate receptors with a Kit-like expression profile across tissues and species were sought. To this end, a manually curated gene expression database was built using NCBI Gene Expression Omnibus (GEO), the largest public repository of microarray data currently containing nearly 400,000 microarrays (Barrett, T. et al. Nucleic Acids Res 35, D760-765 (2007)). A keyword search was performed on individual GEO samples (GSMs) for the term “hematopoietic stem”, yielding 155 human and 81 mouse GSMs. Manual curation was then performed on the resulting human GSMs to annotate those in which the experimental procedures used to purify HSC were not stringent enough, likely resulting in a large proportion of contaminating non-HSC, or those in which the HSC were induced to differentiate. This step identified 25 GSMs as showing high stringency in selection for HSC and 130 of lower stringency. We confirmed that Kit expression in these 25 GSMs is indeed higher than in the remaining 130 (FIG. 2), consistent with both the published literature on Kit expression and the premise behind our curation procedure.

Kit expression is high in stem cell populations such as HSC and low in most mature cell-types. To compare expression profiles across tissues, GSMs related to a representative subset of mature cell-types were included in the database, found using “lung”, “kidney” and “adipocyte” as search terms (see above) in GEO. As expected from prior studies, Kit expression was indeed higher in HSC than kidney and adipocyte cells (FIG. 2). Kit expression was also high in lung cells, which is consistent with analysis of fetal lung cells (Su, A. I. et al. Proc Natl Acad Sci U S A 99, 4465-4470 (2002)). Taken together, these observations attest to the quality of the curated database we assembled from a public repository.

To predict Kit-activated receptors using our database, a three-step method was then employed (FIG. 1). First, an initial list of candidate receptors was generated comprising the 26 genes encoding known Type I cytokine receptors, since the two known Kit-activated receptors, EpoR and IL-7R, belong to this receptor family. The similarity between the expression profiles of Kit and these genes encoding candidate receptors was then determined using hierarchical clustering of normalized data from our expression database (FIG. 2). The specificity of predictions generated from this clustering analysis was progressively improved using three filters, described below (Table 2).

TABLE 2
Prediction of IL-4R as a Kit-interacting
receptor in hematopoietic cells.

| | Filter 1: | | |
|---|---|---|---|
| Candidate | Kit-like | | |
| Type I | expression | Filter 2: | Filter 3: |
| cytokine | profile | all subunits | cross species |
| receptors | across tissues | expressed | expression |
| IL-6R | + | | |
| GM-CSFR/CSF-2R | + | + | |
| IL-4R | + | + | + |
| IL-3R | + | + | |
| IL-7R | + | + | |
| EpoR | + | + | + |
| IL-13R | + | | |
| IL-12R | | | |
| PrlR | | | |
| IL-2R | | | |
| IL-9R | | | |
| IL-5R | | | |
| GHR | | | |
| CSF-3R | | | |
| IL-15R | | | |

Type I cytokine receptors that comprised the initial candidate list are listed in the first column and results of the three successive specificity filters are shown in the others. Genes admitted by each filter are indicated by a plus sign (+). Filter 1 selects receptors that are represented by genes within the KIT-EPOR-IL2RG clades (FIG. 2), indicating co-expression with Kit across tissues. Filter 2 identifies those receptors for which all subunits are admitted by Filter 1. Filter 3 further restricts this list to receptors that pass filter 2 based on both human and murine data. With the exception of EpoR, which is known to interact with Kit, IL-4R (bold) is the sole receptor, all subunits of which (namely, IL4RA and IL2RG) exhibit a Kit-like expression profile across tissues and species. All three criteria are not satisfied for the other Type I cytokine receptors, suggesting that Kit may interact specifically with IL-4R.

For the first filter, we reasoned that receptors activated by Kit in hematopoietic cells would exhibit expression profiles similar to Kit, being higher in HSC than in mature non-hematopoietic cells. Consistent with this reasoning, genes encoding EpoR (EPOR) and IL-7R (IL7RA and IL2RG), which are known to be activated by Kit during hematopoiesis (in erythroid and thymic progenitors respectively), were indeed found to cluster with Kit (FIG. 2). The smallest clade in the dendrogram from our clustering analysis was then identified that included Kit, EPOR, IL2RG and IL7RA. As the first specificity filter, the search for novel Kit-activating receptors was restricted to the remaining receptors in this clade (Table 2).

Many of the candidate receptors are comprised of subunits encoded by multiple genes, as in the case of IL-7R. Both genes encoding subunits of IL-7R, namely IL7RA and IL2RG, belong to the Kit-EPOR-IL2RG clade in data from murine samples. To prioritize the list of candidate receptors in our second filter, those receptors for which all subunits were represented in the above clade were identified. For instance, of the candidate Type I cytokine receptors, six (IL-2R, IL-4R, IL-7R, IL-9R, IL-15R and IL-21R) share the common gamma (γc) subunit encoded by IL2RG but have distinct alpha subunits. Only two of these receptors meet the above criterion: IL-7R and IL-4R. Similarly, not all receptors containing the subunit encoded by CSF2RB were chosen at this step. Thus, the second specificity filter identified IL-4R (IL4RA and IL2RG), GM-CSFR/CSF-2R(CSF2RA and CSF2RB), and IL-3R (IL-3RA and CSF2RB) as potential Kit interacting receptors (Table 2).

For the third specificity filter, the results of clustering data from mouse cells to those from human cells (FIG. 2) were compared to identify interactions predicted to be conserved across mammals. It was noted that IL7RA did not cluster with Kit in the human data. Nevertheless, as two of the three receptor subunits known to be activated by Kit, EPOR and IL2RG, did cluster with Kit in both mouse and human data, the search was narrowed based on cross-species conservation. Of the original 26 candidate genes, only IL-4R was predicted to interact with Kit in both humans and mice, passing this and the above two filters (Table 2). Thus all subunits of the IL-4R receptor have expression profiles similar to Kit across tissues and species, demonstrating a potential role for IL-4R in Kit-mediated signaling in hematopoietic cells. Interestingly, IL-4R is also expressed in a Burkitt lymphoma cell line and in B cells, in T3M4 cells (pancreatic cancer-cell line), SR cells (large cell immunoblastic lymphoma), RL7 (B cell lymphoid tumor), NCI H322M cells and EKVX cells (non-Small Cell Lung cell lines), HSG cells (Human epithelial submandibular salivary gland), and hs578t cells (breast cancer cell line), indicating that IL-4R, like Kit, may play a role in cancer.

Activation of IL-4R by Kit. To test predictions that Kit interacts with IL-4R, the human megakaryoblastic cell line M07e was used. M07e cells are known to express both Kit and IL-4R (Jahn, T. et al. Blood 110, 1739-1747 (2007); Kanakura, Y. et al. Blood 76, 706-715 (1990)) and are therefore well suited to test the predicted Kit:IL-4R interaction. In addition, M07e cells are easily cultured in large enough numbers for biochemical analysis of receptor activation. Previous studies of Kit:EpoR and Kit:IL-7R interactions have shown that stimulation of erythroid and thymic progenitor cells respectively, with Kit ligand (KL) results in rapid phosphorylation and activation of EpoR and IL-7R in the absence of the cognate ligands Epo and IL-7 (Jahn, T. et al. Blood 110, 1840-1847 (2007); Wu, H. et al. Nature 377, 242-246 (1995)).

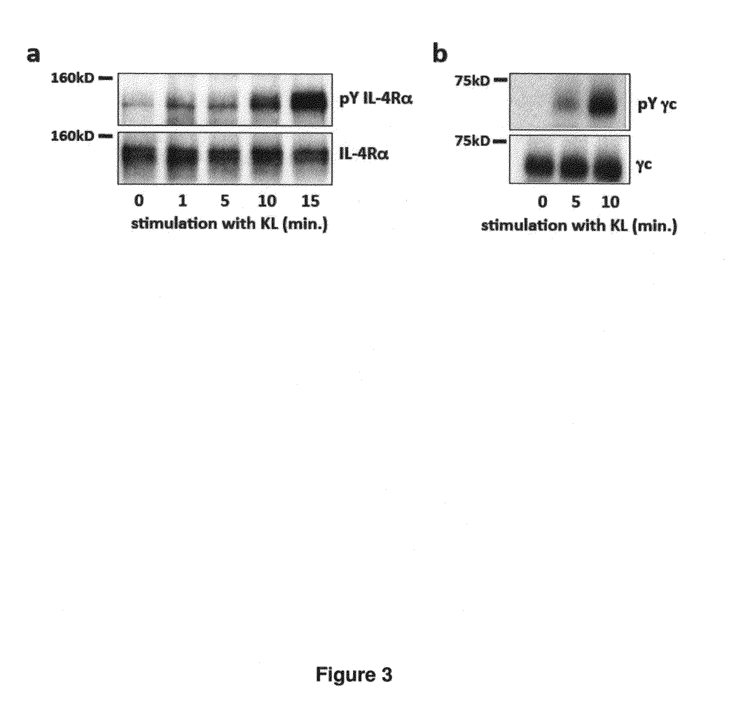

M07e cells were stimulated with KL, which resulted in rapid phosphorylation of Kit, consistent with previous studies (Kuriu, A. et al. Blood 78, 2834-2840 (1991)). Strikingly, both the alpha and gamma subunits of IL-4R, encoded by IL4RA and IL2RG respectively, were also phosphorylated within 5 minutes of KL treatment despite the fact that these cells were not treated with the cognate ligand IL-4 (FIG. 3). This demonstration that Kit signaling can activate IL-4R in cells of hematopoietic origin validates the methodology of applying a guilt-by-association on publicly available microarray data to find a Kit-activated receptor.

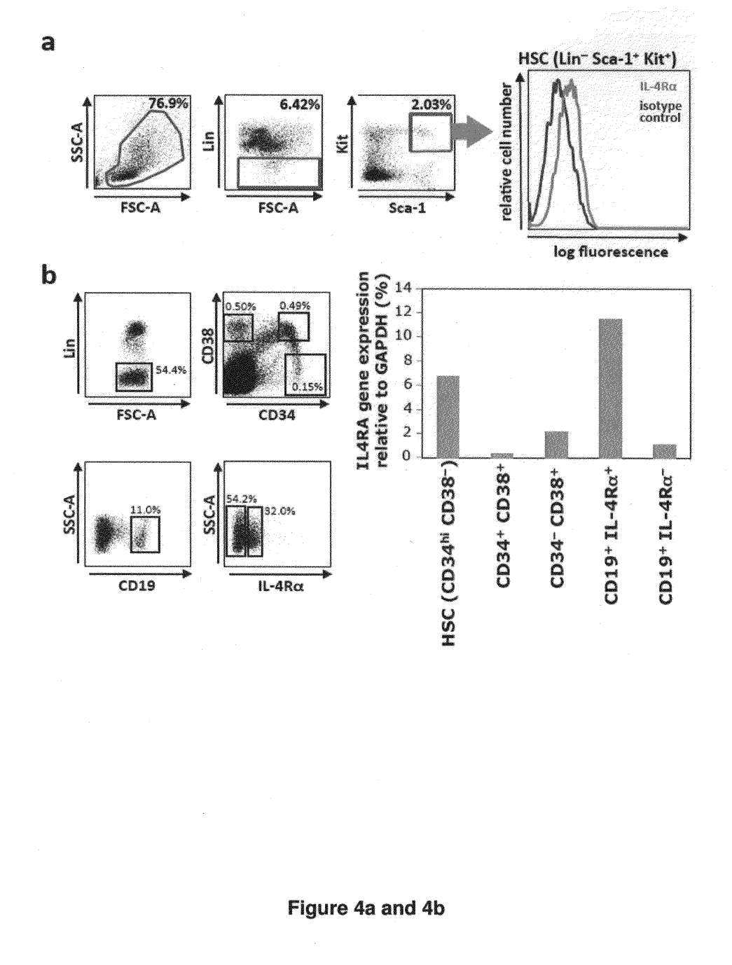

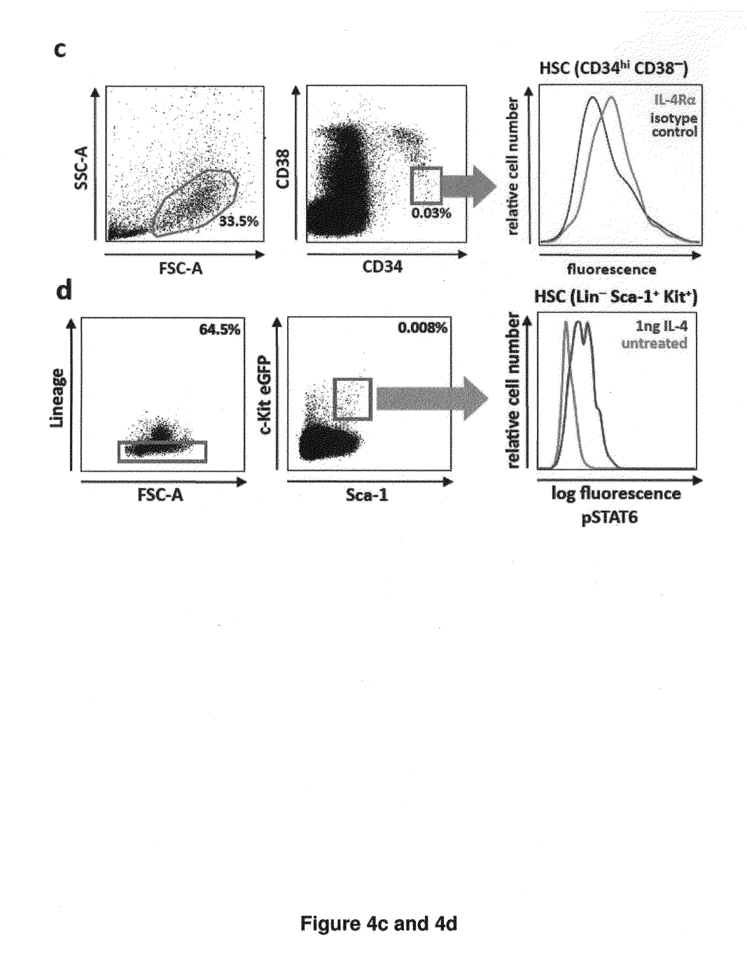

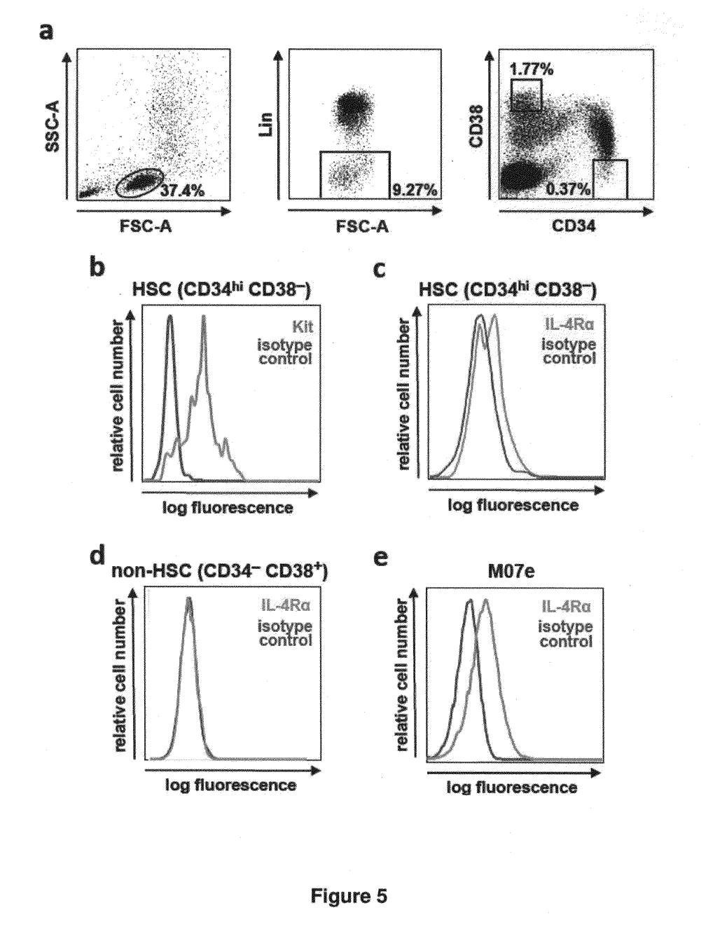

Expression of functional IL-4R on the surface of HSC. Early hematopoietic progenitors, specifically HSCs, are represented in the hematopoietic samples used in the initial analysis, and it was hypothesized that Kit activates IL-4R in these cells. While expression of IL-4Rα mRNA has been noted in murine HSC from gene expression data (Ramalho-Santos, M. et al. Science 298, 597-600 (2002)), the surface expression of IL-4R protein has not been reported in either murine or human HSC. As surface expression of IL-4R is a prerequisite for the hypothesized Kit:IL-4R interaction, the level of the IL-4Rα subunit of IL-4R on HSC was quantified using flow cytometry. Murine HSC were phenotypically identified on the basis of expression of Kit, Sca-1 and lack of expression of a panel of lineage markers (Lin-Sca-1+Kit+, LSK). IL-4R was found to be expressed on the surface of murine HSC (FIG. 4a), at a level comparable to that observed on M07e cells (FIG. 5).

It has been unclear from previous studies whether human HSC express Kit. Human HSC were obtained based on their Lin⁻CD34^{hi}CD38⁻ phenotype using two independent sources: bone marrow samples and peripheral blood stem cells (PBSC) collected from donors treated with granulocyte colony stimulating factor (G-CSF). The analysis of these samples indicates unequivocally that these cells are indeed Kit-positive (FIG. 5). Using RT-PCR, IL-4Rα mRNA was detected in these cells at approximately 60% of the level in CD19⁺ B-cells that express IL-4R (FIG. 4b). In addition, flow cytometry detected surface expression of IL-4R on at least a subset of these cells (FIG. 4c, FIG. 5), although the level of IL-4R was variable between samples. Taken together, the data indicate that HSC co-express Kit and IL-4R.

Finally, the functionality of IL-4R on HSC was assayed. Activated IL-4R is known to phosphorylate the STAT6 transcription factor in B and T lymphocytes (Hou, J. et al. Science 265, 1701-1706 (1994); Takeda, K. et al. Nature 380, 627-630 (1996)). Using flow cytometry for both surface antigens and intracellular phospho-STAT6, STAT6 phosphorylation was observed in murine HSC in response to IL-4 stimulation (FIG. 4d). Taken together, the above data indicate co-expression of functional IL-4R and Kit on the surface of HSC.

### Discussion

We have developed and implemented a bioinformatics approach, named CORSiTE, to identify and prioritize candidate functional interactions between signal transduction proteins. Using this approach we predicted and successfully validated IL-4R as a novel Kit-activated receptor that functions in HSC.

CORSiTE extends the guilt-by-association heuristic applied to mRNA coexpression, which has been widely used to identify functionally related genes, in three ways that represent advancements over previous work. First, our method incorporates prior knowledge to inform the search for Kit-interacting receptors. The list of initial candidates was restricted to members of the receptor family to which the two known Kit-interacting receptors, EpoR and IL-7R, belong, namely the Type I cytokine receptors family. Second, we searched for and manually curated gene expression samples from a public repository to distinguish those of particular relevance to the signaling process being studied. This curation was able to explain variability between the expression profiles of biological samples that were annotated with the same keyword, illustrating the advantage of such curation in fully utilizing data from public repositories. Third, we applied successive specificity filters to refine the results obtained from our initial coexpression analysis. Thus, our method combines an open-ended search using guilt-by-association with a hypothesis-driven focus using prior knowledge and specificity filters to derive a hierarchy of candidates for experimental validation. The previous identification of Kit-interacting receptors was based on direct hypothesis-driven biochemical analysis and deductive reasoning, which cannot predict novel interactions. In contrast, we have used inductive reasoning and public gene expression data to generate predictions for interactions between signaling components. To our knowledge, CORSiTE is the only such approach described so far for predicting novel signaling targets.

Our data indicates that in response to Kit ligand (KL), Kit trans-activates the IL-4R receptor by physically interacting with and phosphorylating the alpha and gamma subunit of IL-4R in a manner analogous to trans-activation of EpoR in erythroid progenitors and IL-7R in thymic progenitors. This model explains the functional synergy that has been observed in hematopoietic progenitors between KL and IL-4 (Sonoda, Y. et al. Br J Haematol 96, 781-789 (1997)). Previous studies have implicated a receptor containing the common gamma subunit encoded by IL2RG in regulating HSC function (Ohbo, K. et al. Blood 87, 956-967 (1996)). Our studies demonstrate that it is the IL-4R receptor that is responsible for this phenotype. Combined signaling via both Kit and IL-4R may result in activation of a unique combination of transcription factors and their target genes, providing a mechanism for transcriptional responses to KL that are specific to HSC and other hematopoietic progenitors.

Activation by Kit of the IL-4R signaling pathway and its downstream transcriptional program may play an important role in regulating early hematopoiesis. Similar interactions between Kit and other cell type-specific Type I cytokine receptors may underlie the pleiotropic effects of Kit signaling in different cell types and in concert with different cytokines. Observation of such interactions would lend further support to a modular signaling paradigm in which Kit co-opts cell type-specific cytokine signaling pathways in different cell types. Example 2 below demonstrates that IL-4R does indeed mediate cell fate specification and development of cells of the hematopoietic lineage.

Our analysis also identified two receptors, IL-3R and GM-CSFR(CSF-2R), that passed two of the three specificity filters described above. Ligands for these receptors are known to synergize with KL for activation of certain downstream effector molecules (Mantel, C. et al. Blood 88, 3710-3719 (1996) Pearson, M. A. et al. Growth Factors 15, 293-306 (1998)). Furthermore, these two receptors share a common beta subunit encoded by CSF2RB, which has been shown to form a complex with Kit (Lennartsson, J. et al. J Biol Chem 279, 44544-44553 (2004)). Taken together, these findings highlight the sensitivity and specificity of CORSiTE in identifying Kit-interacting receptors.

The unexpected finding of functional IL-4R on HSC extends the range of hematopoietic cells that utilize the IL-4R/STATE signaling pathway, which has previously been associated with humoral immunity (Hou, J. et al. Science 265, 1701-1706 (1994)). Although previous studies of IL-4R-deficient mice had showed no overt abnormalities, IL-4 stimulates hematopoiesis in vitro and in vivo (Broxmeyer, H. E. et al. Immunity 16, 815-825 (2002); Broxmeyer, H. E. et al. J Immunol 141, 3852-3862 (1988)). The co-expression of IL-4R and Kit in HSC suggests that this hematopoietic phenotype may result from abnormalities in HSC signaling, not just in committed progenitors. Our findings open up new avenues for in vivo studies of the role of IL-4 signaling in HSC.

Our discovery of the Kit:IL-4R interaction was based entirely on publicly available gene expression data. These data represent experiments carried out in different labs, by different individuals, on different samples, at different times and using different technologies, raising concerns about their overall consistency and relevance to the annotated experiments. Our use of such data to predict and validate a novel signal transduction interaction demonstrates their underlying coherence when combined with manual curation. Given the labor, time and expense involved in experimental validation, our methods offer a strategy for prioritizing candidate signaling molecules by using the vast and rapidly growing body of publicly available data.

CORSiTE, the bioinformatics methodology we have developed, can be applied to other datasets to predict not only biochemical interactions but also functional interactions between signaling proteins based on conserved co-expression of multiple subunits across relevant cell types. In addition, the framework we have described could be easily extended to the discovery and validation of novel interactions between entire signaling pathways based on conserved col 6 expression of pathway members across tissues and organisms.

### Example 2

Previously, IL-4R^{-/-} mice were shown to have increased resistance to Leishmania major infection, impaired alternative macrophage activation, progressive weight loss begins 6 weeks after S. mansoni infection, liver inflammation, liver fibrosis, increased levels of IgG2 in response to N. brasiliensis infection, lower circulating levels of IgE, lower circulating levels of IgE, impaired II-4 induced CD4+ T cell proliferation, increased IgE levels, airway hyperresponsiveness to methacholine, decreased persistence of Th2 cells as indicated by II-4, II-5, and II-13 production, progressive weight loss begins 6 weeks and increased mortality after S. mansoni infection, airway inflammation following chronic allergen challenge, and increased in eosinophils and monocytes found in lung parenchyma.

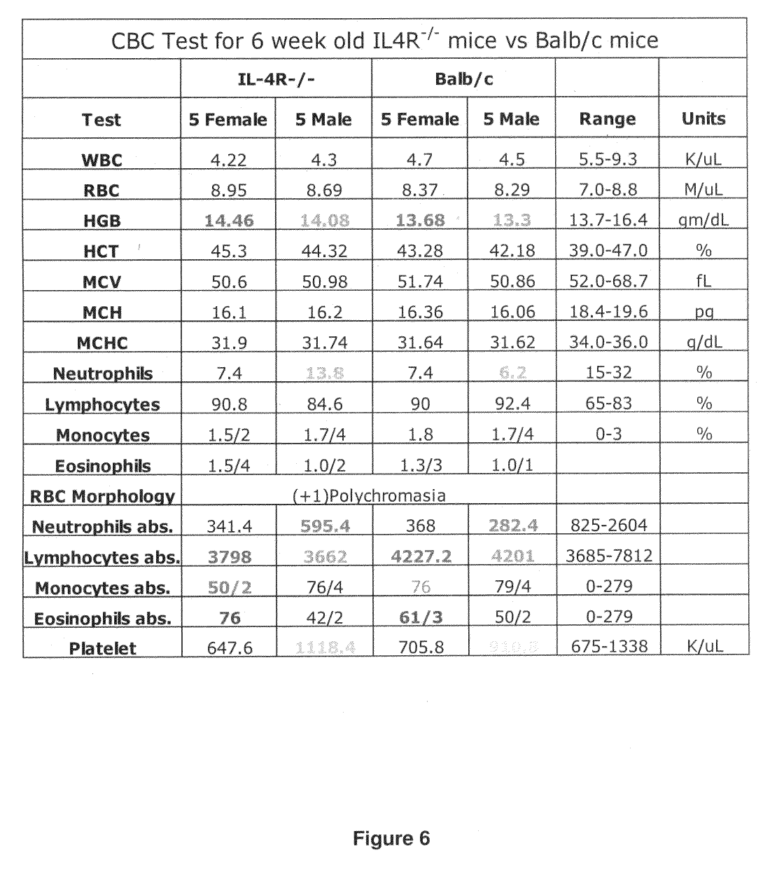

The representation of different hematopoietic cell types in IL-4R deficient mice was analyzed. As demonstrated in FIG. 6, by 5-6 weeks of age, elevated numbers of erythrocytes, myeloid-lineage cells such as neutrophils, monocytes, and eosinophils, and of megakaryocytes (e.g. by platelet count). In contrast, reduced numbers of lymphocytes are observed. Thus, IL-4R appears to mediate cell fate specification and development of cells of the hematopoietic lineage, by promoting the specification of lymphocytes and/or suppressing the specification of non-lymphocytic cell types. This data establishes the principle that IL-4R is a target for modulation of blood cell production either by inhibitory or activating agents. Furthermore, in view of the interaction between Kit and IL-4R demonstrated in Example 1, it is expected that such modulatory agents may include agents that modulate Kit signaling.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of modulating Kit signaling in a cell, comprising the steps of:
contacting a Kit⁺IL-4R⁺ cell with an effective amount of an IL-4R modulating agent under conditions that promote cell survival, and
measuring Kit signaling as a function of IL-4R signaling,
wherein an effective amount of an IL-4R modulating agent to reduce IL-4R signaling reduces Kit signaling, and an effective amount of an IL-4R modulating agent to promote IL-4R signaling promotes Kit signaling.

2. The method according to claim 1, wherein the IL-4R modulating agent reduces IL-4R signaling.

3. The method according to claim 2, wherein the contacting occurs in vitro.

4. The method according to claim 2, wherein the contacting occurs in vivo in an individual.

5. The method according to claim 4, wherein the individual has an anemia, neutropenia, monocytopenia, eosinopenia, thrombocytopenia, lymphoma, and/or leukemia.

6. The method according to claim 5, wherein the lymphoma is a B-cell lymphoma.

7. The method according to claim 5, wherein the leukemia is acute lymphoblastic leukemia (ALL).

8. The method according to claim 4, wherein the number of erythrocytes, neutrophils, monocytes, eosinophils, and platelets in the individual is increased and the number of lymphocytes in the individual is decreased relative to the number of erythrocytes, neutrophils, monocytes, eosinophils, platelets, and lymphocytes in the individual prior to the contacting.

9. The method according to claim 4, wherein the method further comprises contacting the Kit⁺IL-4R⁺ cell with a Kit inhibitor.

10. The method according to claim 9, wherein the method provides for enhanced responsiveness to the Kit inhibitor relative to contacting the Kit⁺IL-4R⁺ cell with Kit inhibitor in the absence of the agent that reduces IL-4R signaling.

11. The method according to claim 1, wherein the IL-4R modulating agent promotes IL-4R signaling.

12. The method according to claim 11, wherein the contacting occurs in vitro.

13. The method according to claim 11, wherein the contacting occurs in vivo in an individual.

14. The method according to claim 13, wherein the individual has polycythemia, an infection, atopy, and/or lymphocytopenia.

15. The method according to claim 13, wherein the number of erythrocytes, neutrophils, monocytes, eosinophils, and platelets in the individual is decreased and the number of lymphocytes in the individual is increased relative to the numbers of erythrocytes, neutrophils, monocytes, eosinophils, platelets, and lymphocytes in the individual prior to the contacting step.

16. The method according to claim 11, wherein the method further comprises contacting the Kit⁺IL-4R⁺ cell with a Kit activator.

17. The method according to claim 16, wherein the method provides for enhanced responsiveness to the Kit activator relative to contacting the Kit⁺IL-4R⁺ cell with Kit activator in the absence of the agent that promotes IL-4R signaling.

18. A method of modulating IL-4R signaling in a cell, comprising:
contacting a Kit⁺IL-4R⁺ cell with an effective amount of a Kit modulating agent under conditions that promote cell survival; and
measuring IL-4R signaling,
wherein an effective amount of a Kit modulating agent to reduce Kit signaling reduces IL-4R signaling, and an effective amount of a Kit modulating agent to promote Kit signaling promotes IL-4R signaling.

19. The method according to claim 18, wherein the Kit modulating agent reduces Kit signaling.

20. The method according to claim 19, wherein the contacting occurs in vitro.

21. The method according to claim 19, wherein the contacting occurs in vivo in an individual.

22. The method according to claim 21, wherein the number of erythrocytes, neutrophils, monocytes, eosinophils, and platelets in the individual is increased and the number of lymphocytes in the individual is decreased relative to the numbers of erythrocytes, neutrophils, monocytes, eosinophils, platelets, and lymphocytes in the individual prior to the contacting step.

23. The method according to claim 18, wherein the Kit modulating agent promotes Kit signaling.

24. The method according to claim 23, wherein the contacting occurs in vitro.

25. The method according to claim 23, wherein the contacting occurs in vivo.

26. The method according to claim 25, wherein the number of erythrocytes, neutrophils, monocytes, eosinophils, and platelets in the individual is decreased and the number of lymphocytes in the individual is increased relative to the numbers of erythrocytes, neutrophils, monocytes, eosinophils, platelets, and lymphocytes in the individual prior to the contacting.

27. A method of enhancing responsiveness of cancer cells to a Kit inhibitor, the method comprising:
contacting Kit⁺IL-4R⁺ cells with an effective amount of a Kit inhibitor and an effective amount of an IL-4R inhibitor under conditions that promote cell survival; and
measuring the survival, proliferation, and/or migration of the Kit⁺IL-4R⁺ cells
wherein the survival, proliferation, and/or migration of the Kit⁺IL-4R⁺ cells is reduced relative to survival, proliferation, and/or migration of Kit⁺IL-4R⁺ cells contacted with a Kit inhibitor in the absence of an IL-4R inhibitor.

28. A method of augmenting the proliferation of stem cells, the method comprising:
contacting a population comprising Kit⁺IL-4R⁺ stem cells with an effective amount of a Kit activator and an effective amount of an IL-4R activator under conditions that promote cell survival; and
measuring the number of cells in the population,
wherein the number of cells in the population is elevated relative to the number cells in a population comprising Kit⁺IL-4R⁺ stem cells that are contacted with a Kit activator in the absence of an IL-4R activator.

29. A method of screening candidate agents for activity in reducing Kit signaling, comprising:
contacting a population of Kit⁺IL-4R⁺ cells with a candidate agent under conditions that promote cell survival, and
measuring Kit signaling as a function of IL-4R activity,
wherein a reduction in IL-4R activity in the contacted population relative to a Kit⁺IL-4R⁺ population that has not be contacted with the agent indicates that the candidate agent is effective in reducing Kit signaling.
